(19) United States
(12) Patent Application Publication
(10) Pub. No.: US 2024/0260856 A1
(43) Pub. Date: Aug. 8, 2024

(54) SYSTEMS AND METHODS FOR AIR CONDUCTION AND BONE CONDUCTION TESTING USING AUDIOMETER HEADSETS

(71) Applicant: Creare LLC, Hanover, NH (US)

(72) Inventors: Brian GRAYBILL, Hanover, NH (US); Cyle ZIEGLER, Anchorage, AK (US); James A. NORRIS, Lebanon, NH (US); Odile H. CLAVIER, Hanover, NH (US); Todd THURSTON, Lebanon, NH (US); William FINGER, Hanover, NH (US)

(21) Appl. No.: 18/176,947

(22) Filed: Mar. 1, 2023

Related U.S. Application Data
(60) Provisional application No. 63/483,642, filed on Feb. 7, 2023.

(51) Int. Cl. A61B 5/12; A61F 11/14

(52) U.S. Cl. CPC A61B 5/121; A61F 11/14; A61B 5/0022

(57) ABSTRACT

Disclosed are methods, systems, and non-transitory computer readable memory for hearing testing. In some cases, headsets, interface devices, and an administrator device may wirelessly communicate to perform hearing testing of a user. In some cases, the hearing testing may be fit or hearing testing. In some cases, the headset may be configured to perform hearing testing using air conduction or bone conduction.

### CROSS-REFERENCE TO RELATED APPLICATION(S)

This application claims the benefit of priority under 35 U.S.C. §§ 120 and 119(e) of U.S. provisional application No. 63/483,642, filed Feb. 7, 2023, the entire contents of which is incorporated herein by reference.

### TECHNICAL FIELD

Various aspects of the present disclosure relate generally to systems and methods for hearing testing and, more particularly, to wirelessly connected systems for hearing testing.

### BACKGROUND

In the general public, hearing loss is the third most common chronic physical condition—more prevalent than diabetes or cancer. 14.1% (27.7 million) of adults aged 20-69 years of age have unilateral or bilateral hearing impairment that impacts speech understanding and communication. Many forms of hearing loss are treatable medically but many individuals do not benefit from treatment in part because of a lack of access to diagnostic quality testing. A major limiter to accurate, diagnostic quality hearing tests is the need for expensive infrastructure, i.e., sound-proofed booths, and audiologists with specialized training in conducting exams, which are traditionally conducted in person and one-on-one. Traveling to facilities that offer hearing health assessment is often too great a barrier for many rural and underserved populations.

Moreover, occupational hearing loss is one of the most common work-related illnesses. These industries typically require hearing conservation programs similar to that in the military. Research has demonstrated that workers who wear their earplugs are at increased risk for developing permanent hearing loss. Although several fit-test systems have been developed, they require a trained administrator to operate and are often only compatible with a single manufacturer's earplugs.

Furthermore, noise induced hearing loss and tinnitus are the two most common permanent injuries suffered by service members in the military. The need for advancements in hearing loss prevention strategies and evidence-based auditory fitness-for-duty medical evaluations are both major readiness issues. Despite recent advances in hearing protection technologies, service members often have limited access to these technologies, are not effectively trained to use them properly, or are reluctant to wear them at all due to concerns that hearing protection will limit their situational awareness and thus their effectiveness and survivability in combat. Furthermore, many of the clinical tools and methods used to assess and document the hearing health of our service members are outdated and inefficient, requiring service members to report to a clinic with a sound attenuating booth.

The present disclosure is directed to overcoming one or more of these above-referenced challenges.

### SUMMARY OF THE DISCLOSURE

According to certain aspects of the disclosure, systems, methods, and computer readable memory are disclosed for hearing testing.

In some cases, a system for hearing testing and fit testing, the system may include: a headset, wherein the headset includes: a support structure; and a pair of ear cups connected by the support structure. In some cases, each of the pair of ear cups may include: a cushion configured to seal an interior of the ear cup from an exterior of the ear cup when worn by a user; an audiometer stack disposed in the interior of the ear cup; and an ear cup attenuating structure surrounding the audiometer stack and being connected to the cushion. In some cases, the ear cup attenuating structure may be configured to provide attenuation comparable to that of a sound booth. In some cases, the system may include an interface device configured to wirelessly connect to at least a first audiometer stack of the pair of ear cups. In some cases, the system may be configured to: output a first interface, the first interface including a first interface element configured to be selectable by a first input to start a hearing test; determine that a first input has selected the first interface element; in response to determining the first input has selected the first interface element, instruct at least the first audiometer stack to perform a first series of stimuli; receive a first set of one or more user responses to at least a first subset of the first series of stimuli; based on the first set of one or more user responses, determine a hearing test result of the hearing test, the hearing test result including at least a measure of a hearing ability of the user; output a second interface configured to indicate a transition to a hearing protector fit test, the second interface including at least a second interface element configured to be selectable by a second input to start the hearing protector fit test; determine a second input has selected the second interface element; in response to determining the second input has selected the second interface element, instruct at least the first audiometer stack to perform a second series of stimuli; receive a second set of one or more user responses for at least a second subset of the second series of stimuli; and based on the second set of one or more user responses, determine a fit test result of the hearing protector fit test, the fit test result including an indication of a hearing attenuation achieved by a hearing protector as worn by the user during the hearing protector fit test.

In some cases, a method for hearing testing and fit testing, may include: output a first interface, the first interface including a first interface element configured to be selectable by a first input to start a hearing test; determine that a first input has selected the first interface element; in response to determining the first input has selected the first interface element, instruct at least a first audiometer stack of a pair audiometer stacks to perform a first series of stimuli; receive a first set of one or more user responses for at least a first subset of the first series of stimuli; based on the first set of one or more user responses, determine a hearing test result of the hearing test, the hearing test result including at least a measure of a hearing ability of the user; output a second interface configured to indicate a transition to a hearing protector fit test, the second interface including at least a second interface element configured to be selectable by a second input to start the hearing protector fit test; determine a second input has selected the second interface element; in response to determining the second input has selected the second interface element, instruct at least the first audiometer stack to perform a second series of stimuli; receive a second set of one or more user responses for at least a second subset of the second series of stimuli; and based on the second set of one or more user responses, determine a fit test result of the hearing protector fit test, the fit test result including an indication of a sound attenuation achieved by a hearing protector as worn by the user during the hearing protector fit test.

In some cases, a system for bone conduction hearing or fit testing may include: a headset. In some cases, the headset may include: a support structure; a pair of ear cups connected by the support structure; and a bone conductor configured to be mounted to a head of the user and electronically connected to a first audiometer stack of the pair of ear cups, wherein a second audiometer stack of the pair of ear cups is electronically connected to the first audiometer stack. In some cases, each of the pair of ear cups include: a cushion configured to seal an interior of the ear cup from an exterior of the ear cup when worn by a user; an audiometer stack disposed in the interior of the ear cup; and an ear cup attenuating structure surrounding the audiometer stack and being connected to the cushion. In some cases, the ear cup attenuating structure may be configured to provide attenuation comparable to that of a sound booth. In some cases, the system may include interface device configured to wirelessly connect to the first audiometer stack and transmit instructions to and receive data from the first audiometer stack. In some cases, the first audiometer stack may include: a first analog output, a second analog output, and a third analog output, the first analog output drives a first speaker associated with the first audiometer stack, the second analog output drives a second speaker associated with the second audiometer stack, the third analog output drives the bone conductor, and the third analog output is different than the first analog output and the second analog output.

In some cases, a method for hearing testing and fit testing may include: wirelessly connect an interface device to a first audiometer stack of a headset, to thereby transmit instructions to and receive data from the first audiometer stack; transmit an instruction message to the first audiometer stack of the headset, the instruction message indicating an instruction to start a hearing or fit test of a user, wherein the first audiometer stack includes a first analog output, a second analog output, and a third analog output; in accordance a firmware of the first audiometer stack, drive, via the first analog output, a first speaker associated with the first audiometer stack and the first ear cup; in accordance the firmware of the first audiometer stack, drive, via the second analog output, a second speaker associated with the second audiometer stack and the second ear cup; and in accordance the firmware of the first audiometer stack, drive, via the third analog output, a bone conductor of the headset. In some cases, the first audiometer stack of the headset is associated with a first ear cup of a pair of ear cups of the headset, a second audiometer stack of the headset is associated with a second ear cup of the pair of ear cups of the headset, the second audiometer stack is electronically connected to the first audiometer stack. In some cases, each of the pair of ear cups include: a cushion configured to seal an interior of the ear cup from an exterior of the ear cup when worn by a user, and an ear cup attenuating structure surrounding a respective audiometer stack and being connected to the cushion. In some cases, the ear cup attenuating structure may be configured to provide attenuation comparable to that of a sound booth. In some cases, the bone conductor is configured to be mounted to a head of the user and electronically connected to the first audiometer stack. In some cases, the third analog output is different than the first analog output and the second analog output.

In some cases, a system for hearing or fit testing may include: at least one headset. In some cases, the least one headset includes a first headset. In some cases, the first headset may include: a support structure; and a pair of ear cups connected by the support structure. In some cases, each of the pair of ear cups include: a cushion configured to seal an interior of the ear cup from an exterior of the ear cup when worn by a user; and an audiometer stack disposed in the interior of the ear cup; and an ear cup attenuating structure surrounding the audiometer stack and being connected to the cushion. In some cases, the ear cup attenuating structure is configured to provide attenuation comparable to that of a sound booth. In some cases, the system may include at least one interface device, wherein the least one interface device includes a first interface device configured to wirelessly connect to at least a first audiometer stack of the first headset. In some cases, the system may include an administrator device configured to wirelessly connect to each of the at least one interface device, to thereby transmit instructions to and receive data from each of the at least one headset via the at least one interface device. In some cases, the administrator device may be configured to, in response to receiving an administrator user input on the administrator device, transmit an instruction message, via the first interface device, to the first headset to conduct a hearing or fit test, and the instruction message does not include data indicating frequency of stimuli, amplitude of stimuli, or timing of stimuli.

In some cases, a method for hearing or fit testing may include: wirelessly connect at least one interface device to at least one headset; wherein the least one interface device includes a first interface device configured to wirelessly connect to at least a first audiometer stack of a first headset; wirelessly connect an administrator device to each of the at least one interface device, to thereby transmit instructions to and receive data from each of the at least one headset via the at least one interface device; and in response to receiving an administrator user input on the administrator device, transmit an instruction message, via the first interface device, to the first headset to conduct a hearing or fit test. In some cases, the instruction message does not include data indicating frequency of stimuli, amplitude of stimuli, or timing of stimuli.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### BRIEF DESCRIPTION OF THE DRAWINGS

The accompanying drawings, which are incorporated in and constitute a part of this specification, illustrate various exemplary aspects and together with the description, serve to explain the principles of the disclosed technology.

FIG. 1 depicts an example environment for hearing testing.

FIGS. 2A and 2B depict features of an administrator device and an interface device for hearing testing.

FIGS. 3A-3E depict hearing test or fit test dataflow diagrams between an administrator device, an interface device, and a headset.

FIGS. 4A-4B depict features of a headset.

FIGS. 5A-5C, 6A-6B, and 7A-7B depict features of fit testing.

FIGS. 8A-8C depict features of bone conduction testing.

FIG. 9 depict a flowchart for masking during hearing testing.

FIG. 10 depicts a diagram for accelerated threshold determination.

FIGS. 11A-11E and 12 depict graphic user interfaces on an administrator device and an interface device.

FIG. 13 depicts an example system that may execute techniques presented herein.

### DETAILED DESCRIPTION

Various aspects of the present disclosure relate generally to audiometric testing and, more particularly, to wirelessly connected systems for audiometric testing. In particular, the present disclosure describes features of audiometers and transducers (such as speakers and/or bone vibrators) combined in a headset (referred to as “audiometer headset”), interface devices, and administrator devices for fit or hearing testing using air conduction or bone conduction.

In some cases, an audiometer headset, an interface device, and an administrator device may be wirelessly connected, so as to perform fit or hearing tests using air conduction or bone conduction. The administrator device may be configured to, in response to receiving an administrator user input on the administrator device, transmit an instruction message, via the interface device, to the headset to conduct the hearing or fit test. In some cases, the instruction message may omit certain details required for the test, such as the actual signal that should be presented to the test taker and instead transmit parameters, such as the frequency of the test signal and starting amplitude, for the fit or hearing test, thus avoiding reliance on hardware/software stacks of the administrator device/interface device (e.g., mobile phones or tablets) to output high quality analog signals. In some cases, the instruction message may omit certain details required for the test, such as data indicating frequency of stimuli, amplitude of stimuli, or timing of stimuli for the fit or hearing test.

In some cases, the headset may include a bone conductor. The bone conductor may be configured to be mounted to a head of the user and electronically connected to a first audiometer stack of the pair of ear cups (e.g., inside a first ear cup). A second audiometer stack of the pair of ear cups (e.g., inside a second ear cup) may be electronically connected to the first audiometer stack. The first audiometer stack may include a first analog output, a second analog output, and a third analog output. The first analog output drives a first speaker associated with the first audiometer stack, the second analog output drives a second speaker associated with the second audiometer stack, and the third analog output drives the bone conductor. The third analog output may be different than the first analog output and the second analog output. Thus, bone conduction testing may be automatically conducted using audiometer headsets, interface devices, and administrator devices connected wirelessly.

In some cases, the audiometer headset may automatically perform a hearing and/or fit test, while also providing the user educational information about hearing conservation and/or hearing protectors. In some cases, the audiometer headset may automatically transition from a hearing test and to the fit test. Generally, the interface device may provide user responses to the audiometer headset, so that user response times and response indicators (response or no response) for presented stimuli may be tracked by the audiometer headset. Moreover, the audiometer headset may modify a series of stimuli based on user responses to a first set of presentations, thereby efficiently conducting the automatic hearing or fit test. In some cases, the audiometer headset or interface device may automatically interpret results and provide feedback to the person taking the test. For example, if the fit test indicates the person's earplugs are not providing the expected hearing protection, they could be presented with additional instructions on how to properly fit their hearing protectors. In this manner, the audiometer headset, the interface device, and the administrator device may save time and reduce audiologist workloads.

Thus, methods and systems of the present disclosure may be improvements to computer technology and/or audiometer technology.

### Environment

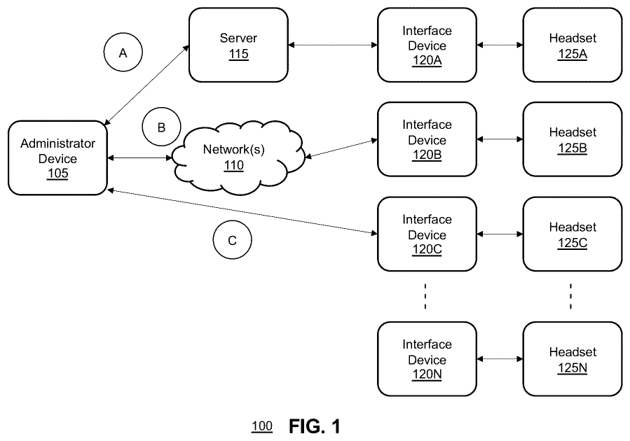

FIG. 1 shows an example environment 100 for hearing testing. The environment 100 may include an administrator device 105, network(s) 110, a server 115, a plurality of interface devices 120A-120N, and a plurality of headsets 125A-125N.

The administrator device 105 may be a personal computing device, such as a cell phone, a tablet, a laptop, or a desktop computer. In some cases, the administrator device 105 may be an extended reality (XR) device, such as a virtual reality device, an argument reality device, a mixed reality device, and the like. In some cases, the administrator device 105 may be associated with a user (e.g., an administrator) of medical services (i.e., an audiologist). The administrator may have a user account associated with the administrator device 105/server 115 that uniquely identifies the administrator.

The network(s) 110 may include one or more local networks, private networks, enterprise networks, public networks (such as the internet), cellular networks, and satellite networks, to connect the various devices in the environment 100. Generally, the various devices of the environment 100 may communicate over network(s) 110 using, e.g., network communication standards that connect endpoints corresponding to the various devices of the environment 100.

The server 115 may (in some cases) manage data and communications between the administrator device 105 and the interface devices 120. In certain aspects, the server 115 may execute a server-side application (e.g., corresponding to a browser program, a mobile application, a desktop application, and the like on the administrator device 105 and/or the interface devices 120) so that instructions, requests, and messages may be stored, managed, and transmitted to relevant endpoints, as discussed herein. The server 115 may store relevant data (as discussed herein) in a data structure. The data structure may be a structured or unstructured database or other data storage system (e.g., time series database). As one would recognize, the data structure may store both data (in various tables or data structures, in different secure methodologies based on sensitivity, etc.) and rules (e.g., medical data, personal data, and the like are stored more securely). In some cases, the server 115 may relay communications between components of environment 100 while also providing functions and/or records to the various components of the environment.

The plurality of interface devices 120A-120N (referred to interface device 120 for ease of reference hereinafter) may be a personal computing device, such as a cell phone, a tablet, a laptop, or a desktop computer. In some cases, the interface device 120 may be an extended reality (XR) device, such as a virtual reality device, an argument reality device, a mixed reality device, and the like. In some cases, the interface device 120 may be associated with a user (e.g., a patient) of medical services or the interface device 120 may be used by various users of an organization. The user may have a user account (or at least records stored in association with a user ID) associated the server 115 that uniquely identifies the user. In some cases, the administrator device 105 may combined with an interface device 120—that is, a patient may use the administrator device, or an administrator may use the interface device. In this case, the dataflows depicted herein still apply, but are passed between applications residing on a same device and not over wireless communication.

The plurality of headsets 125A-125N (referred to headset 125 for ease of reference hereinafter) may be headsets configured to provide hearing testing outside (e.g., not enclosed by) a sound-proofed booth. As discussed herein, the headset 125 may include a support structure (e.g., a headband) and a pair of ear cups connected by the support structure. Each of the pair of ear cups may include: a cushion, an audiometer stack, and an ear cup attenuating structure. The cushion may be configured to seal an interior of the ear cup from an exterior of the ear cup when worn by a user. The audiometer stack may be disposed in the interior of the ear cup. The ear cup attenuating structure may surround the audiometer stack and be connected to the cushion. The ear cup attenuating structure may be configured to provide attenuation comparable to that of a sound booth. Additional details of the headset 125 as discussed herein.

In some cases, a headset 125 may connect to a single interface device 120. In some cases, a single interface device 120 may connect to one or more headsets 125. Generally, headsets 125 may connect to interface devices using a low energy communication standard (e.g., Bluetooth). Thus, the headsets 125 and the interface devices 120 may transmit parameters over Bluetooth low energy, such as (1) a frequency of the test signal and starting amplitude, or (2) only a test indicator (e.g., first indicator corresponds to hearing test, second indicator corresponds to fit test, and the like), while omitting certain details required for the test, such as the actual signal that should be presented to the test taker for the fit or hearing test. In this manner, the system may avoid reliance on hardware/software stacks of the administrator device 105/interface device 120 to output high quality analog signals.

In some cases, the administrator device 105 and one or more interface devices 120 may connect via a first route A. For instance, the first route A may connect the administrator device 105 to the server 115, and the server 115 to the one or more interface devices 120. For instance, each connection to the server 115 may be securely encrypted across the network(s) 110 (e.g., the internet).

In some cases, the administrator device 105 and one or more interface devices 120 may connect via a second route B. For instance, the second route B may connect administrator device 105 to the one or more interface devices 120 via the network(s) 110. For instance, each connection across the network(s) 110 may be securely encrypted across the network(s) 110 (e.g., a local network, a self-broadcast WIFI network from the administrator device 105).

In some cases, the administrator device 105 and one or more interface devices 120 may connect via a third route C. For instance, the third route C may connect the administrator device 105 and the one or more interface devices 120 via Standard

Bluetooth, Low energy Bluetooth, or other low energy communication standard (e.g., Zigbee or other wireless personal area networks (WPANs)).

In some cases, an administrator device 105 and sets of interface devices 120 may be configured to only one route (e.g., Route C) for security reasons. In some cases, an administrator device 105 and sets of interface devices 120 may be configured to use any available route (e.g., selecting a fastest route, a highest bandwidth, a lowest latency, and the like) of routes A, B, or C.

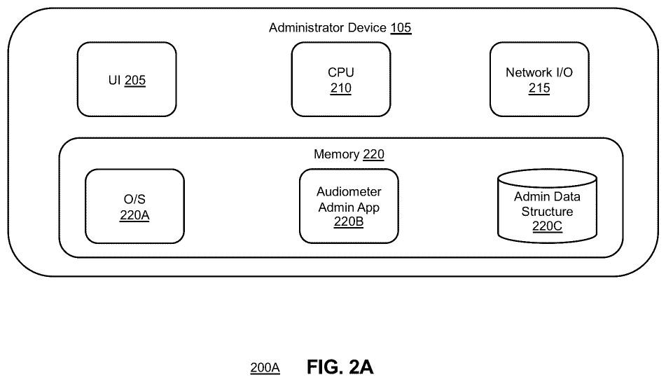

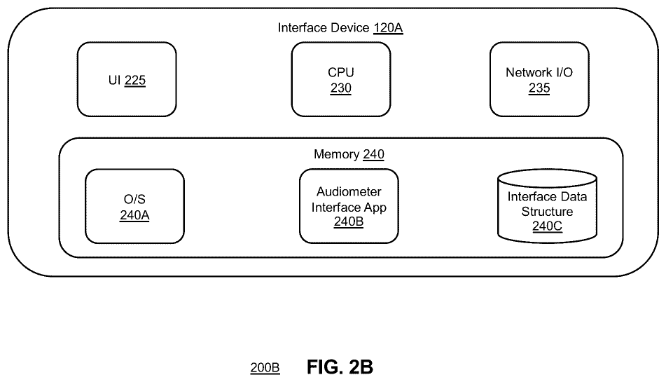

FIGS. 2A and 2B depict features of an administrator device 105 and an interface device 120 for hearing testing. The features of the administrator device 105 and the interface device 120 of FIGS. 2A and 2B may apply to FIGS. 1, 3A-3E, 4A-4B, 5A-5C, 6A-6B, 7A-7B, 8A-8C, 9, 10, 11A-11E, 12, and 13.

FIG. 2A depicts features of an administrator device 105. The administrator device 105 may include a user interface 205 (UI 205), a central processing unit 210 (CPU 210), network input/output 215 (network I/O 215), and a memory 220. The memory 220 may include at least an operating system 220A (O/S 220A), an administrator application 220B (audiometer admin app 220B), and administrator data structure 220C (admin data structure 220C). The administrator device 105 may operate as an administrator interface to control, monitor, and process fit and hearing tests for one or a plurality of users by transmitting instructions to one or a plurality of headsets 125, via interface devices 120, and receiving data/messages from the one or a plurality of headsets 125, via the interface devices 120.

The UI 205 may include a touch display, display, a microphone, a speaker, and/or software or hardware buttons, switches, dials, and the like. The network I/O 215 may one or more network cards to send and receive communications to, e.g., the interface device 120 and/or the network(s) 110.

The memory 220 may store instructions (e.g., software code) for the O/S 220A and the audiometer admin app 220B. The memory 220 may also store data for the audiometer admin app 220B in the admin data structure 220C. The O/S 220A may provide access to hardware/software layers of the administrator device 105 and communications layers of the network I/O 215. The audiometer admin app 220B may provide communication management to the server 115 and the interface devices 120, and provide a user interface (via the UI 205) to interact with features discussed herein. For instance, the audiometer admin app 220B may be a mobile application, a desktop application, a browser-side webapp running in a browser to display graphical user interfaces, receive user inputs, and execute functions/communications, as discussed herein. The admin data structure 220C may be a structured or unstructured database or other data storage system (e.g., time series database). As one would recognize, the admin data structure 220C may store both data (in various tables or data structures, in different secure methodologies based on sensitivity, etc.) and rules (e.g., medical data, personal data, and the like are stored more securely). The CPU 210 may execute the instructions to execute the O/S and the at least the audiometer admin app 220B.

FIG. 2B depicts features of an interface device 120. The interface device 120 may include a user interface 225 (UI 225), a central processing unit 230 (CPU 230), network input/output 235 (network I/O 235), and a memory 240. The memory 240 may include at least an operating system 240A (O/S 240A), an audiometer interface application 240B (audiometer interface app 240B), and interface data structure 240C (interface data structure 240C). The interface device 120 may operate as a rely between the administrator device 105 and the headset 125, and operate as a response device for user response to stimulus output by the headset 125.

The UI 225 may include a touch display, display, a microphone, a speaker, and/or software or hardware buttons, switches, dials, and the like. The network I/O 235 may one or more network cards to send and receive communications to, e.g., the administrator device 105, the headset 125, and/or the network(s) 110.

The memory 240 may store instructions (e.g., software code) for the O/S 240A and the audiometer interface app 240B. The memory 240 may also store data for the audiometer interface app 240B in the interface data structure 240C. The O/S 240A may provide access to hardware/software layers of the interface device 120 and communications layers of the network I/O 235. The audiometer interface app 240B may provide communication management to the administrator device 105, the server 115, and the headset 125, and, and provide a user interface (via the UI 225) to interact with features discussed herein. For instance, the audiometer interface app 240B may be a mobile application, a desktop application, a browser-side webapp running in a browser to display graphical user interfaces, receive user inputs, and execute functions/communications, as discussed herein. The interface data structure 240C may be a structured or unstructured database or other data storage system (e.g., time series database). As one would recognize, the interface data structure 240C may store both data (in various tables or data structures, in different secure methodologies based on sensitivity, etc.) and rules (e.g., medical data, personal data, and the like are stored more securely). The CPU 230 may execute the instructions to execute the O/S and the at least the audiometer interface app 240B.

### Hearing Test or Fit Test Dataflows

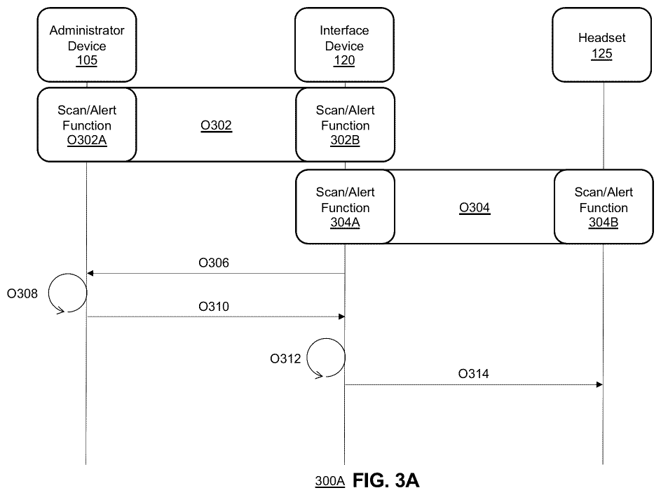

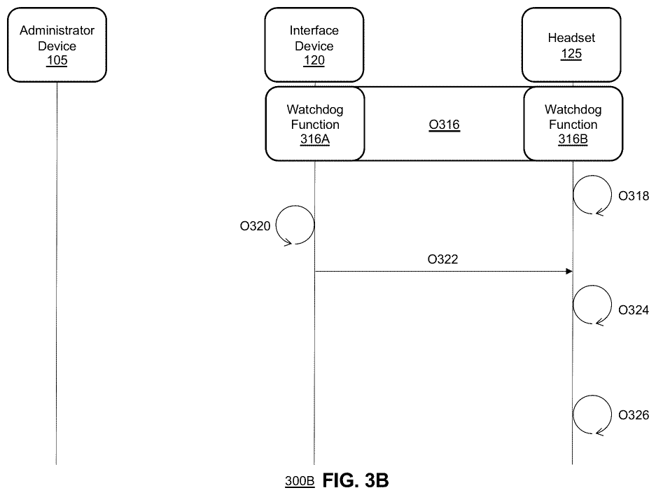

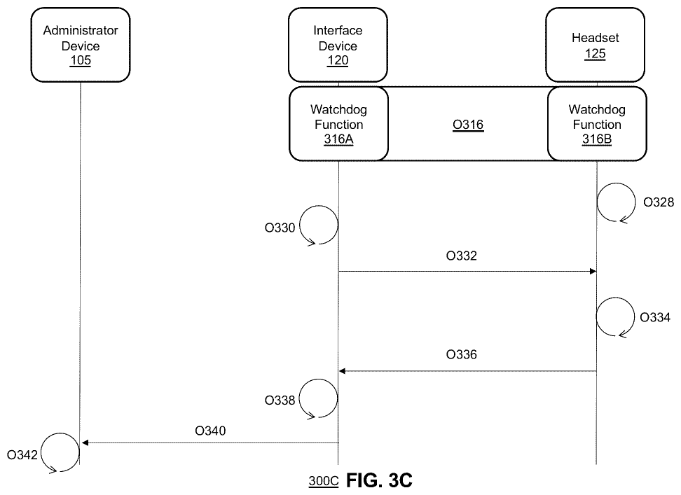

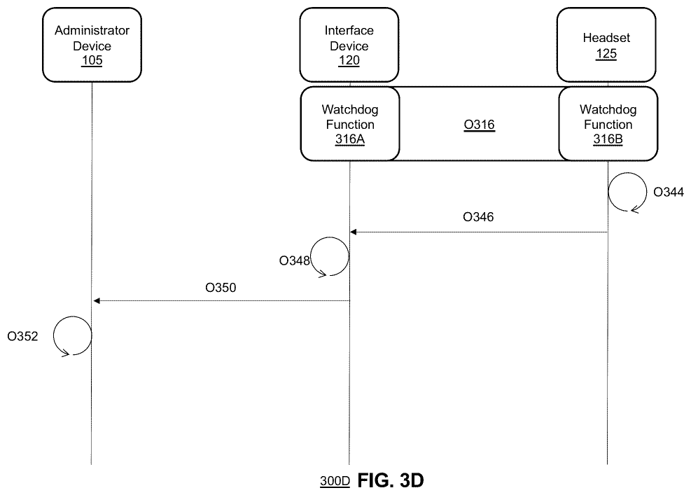

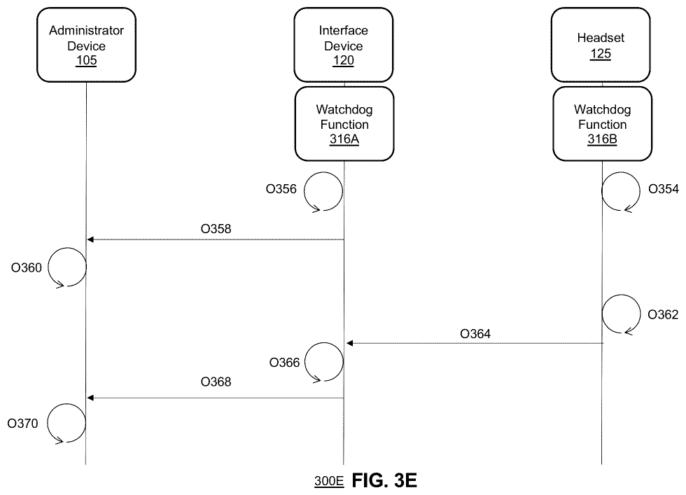

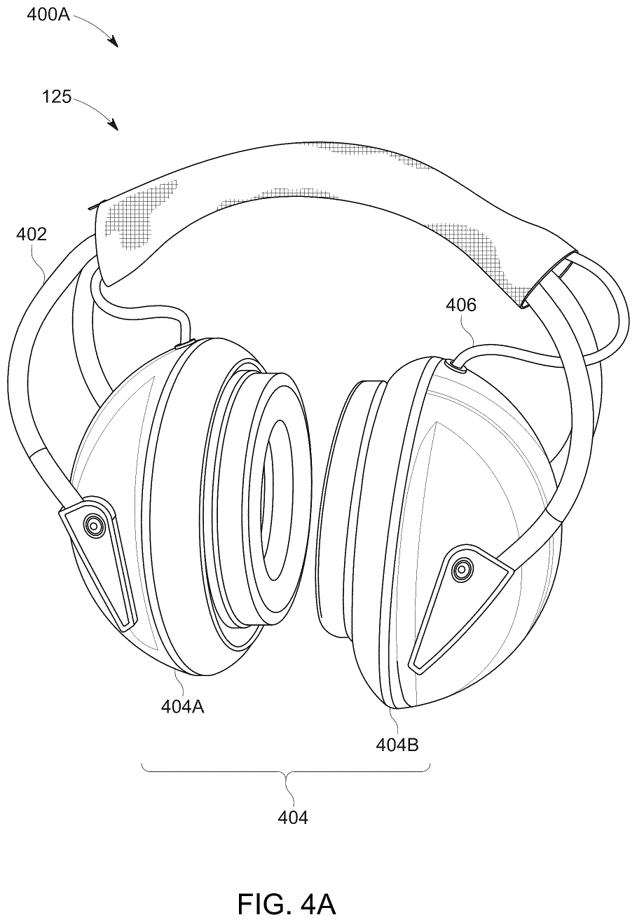

FIGS. 3A-3E depict hearing test or fit test dataflow diagrams 300A, 300B, 300C, 300D, and 300E between an administrator device 105, an interface device 120, and a headset 125. The features of the dataflow diagrams 300A, 300B, 300C, 300D, and 300E between the administrator device 105, the interface device 120, and the headset 125 of FIGS. 3A-3E may apply to FIGS. 1, 2A-2B, 4A-4B, 5A-5C, 6A-6B, 7A-7B, 8A-8C, 9, 10, 11A-11E, 12, and 13.

In operation O302, the administrator device 105 may execute a scan/alert function O302A and the interface device 120 may execute a scan/alert function O302B, to form a first connection. For instance, using Bluetooth, the administrator device 105 may broadcast a signal and the interface device 120 may scan for the broadcast signal to negotiate a connection. In response to forming the first connection, the administrator device 105 and the interface device 120 may pass data and/or instructions.

In operation O304, the headset 125 may execute a scan/alert function O304B and the interface device 120 may execute a scan/alert function O304A, to form a second connection. For instance, using Bluetooth, the headset 125 may broadcast a signal and the interface device 120 may scan for the broadcast signal to negotiate a connection. In response to forming the second connection, the headset 125 and the interface device 120 may pass data and/or instructions.

In operation O306, the interface device 120 may transmit a connection status to the administrator device 105. For instance, the interface device 120 may transmit the connection status to the administrator device 105 after the first and second connections are formed. The connection status may indicate the second connections is formed. In some cases, the connection status may include data about the headset 125 (e.g., a headset ID, headset battery status, etc.).

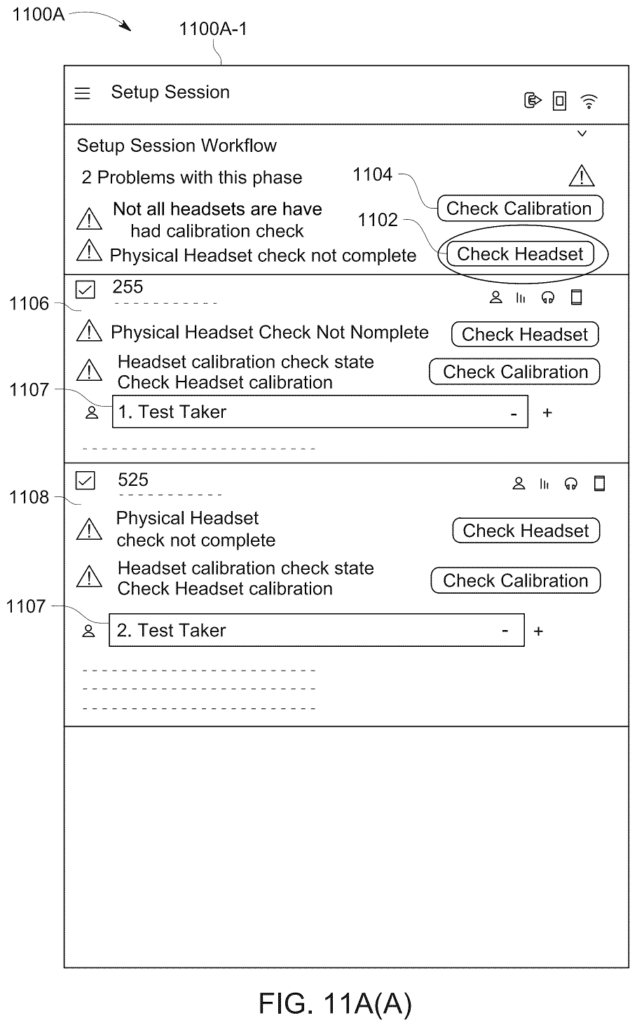

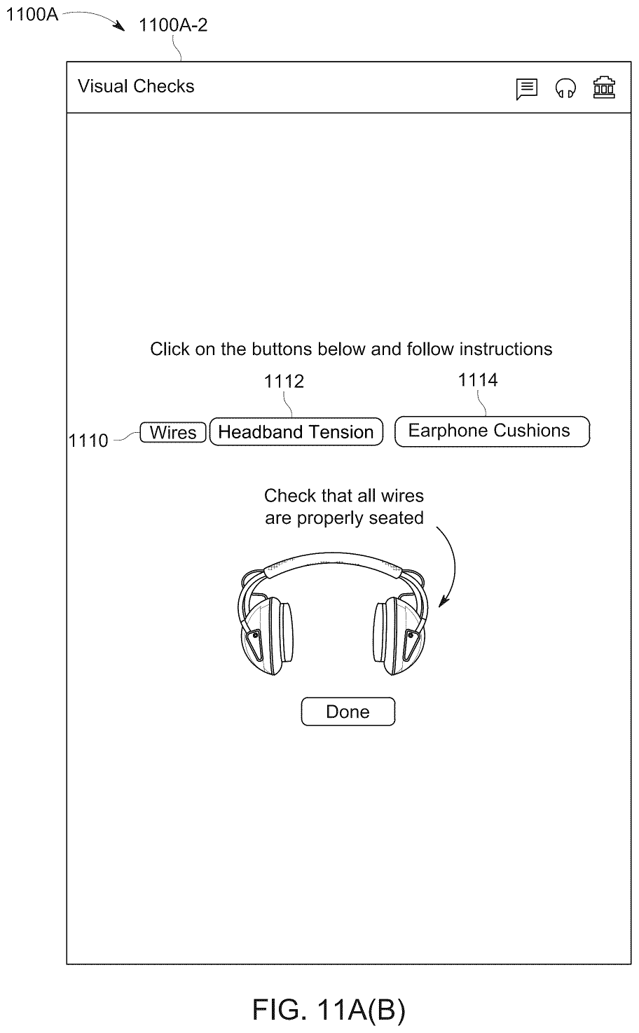

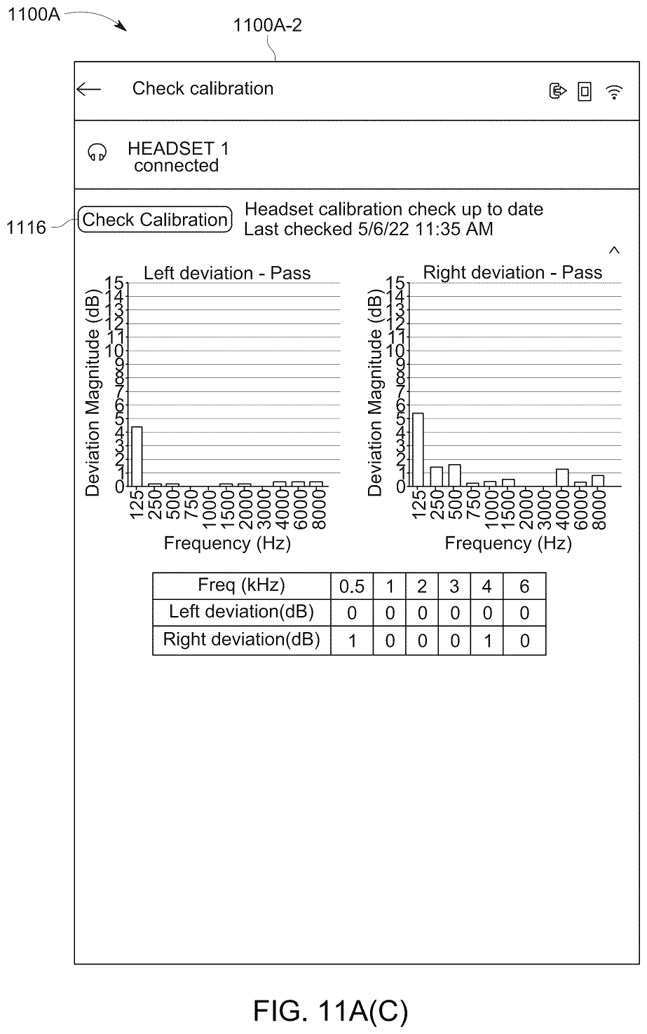

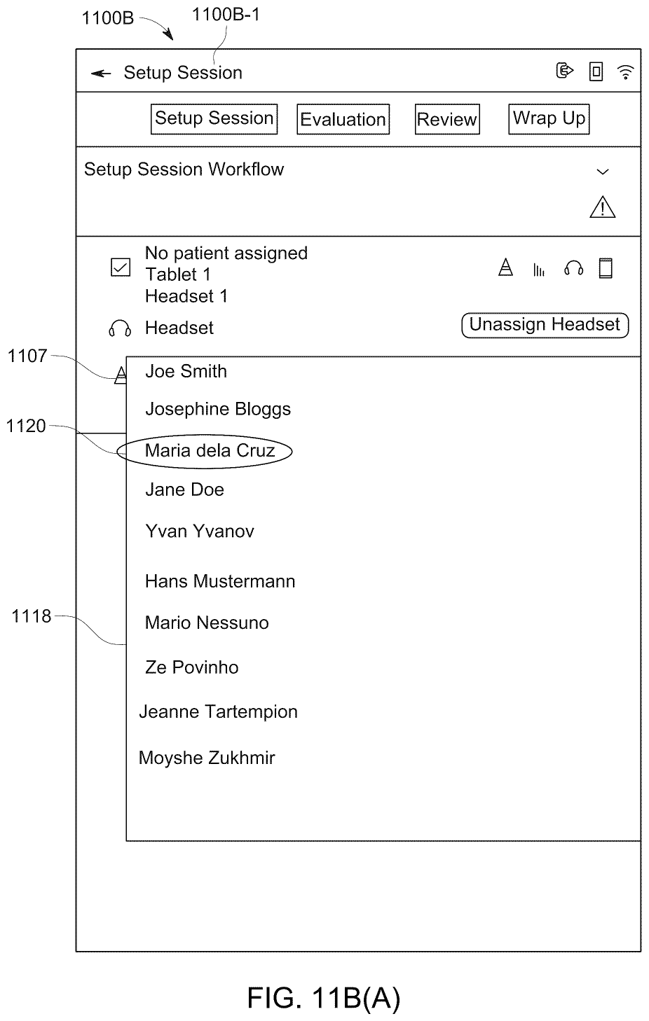

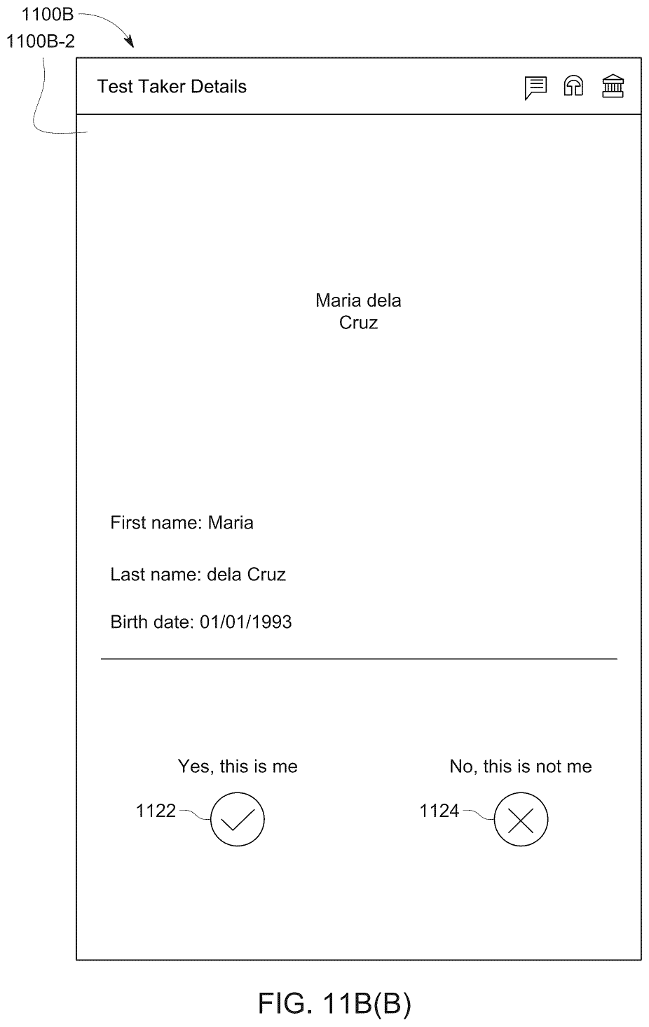

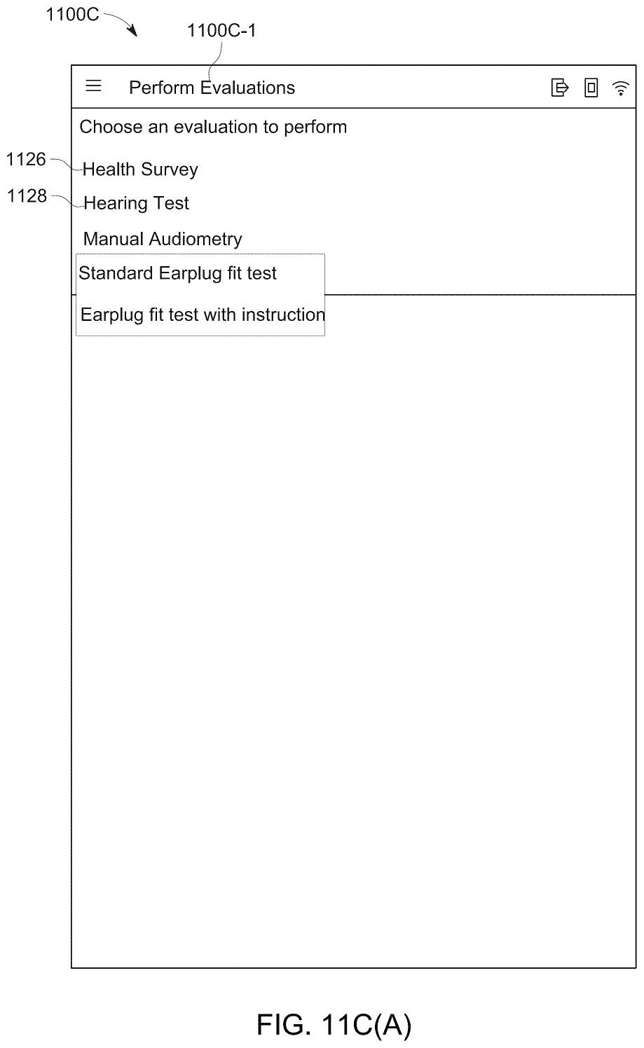

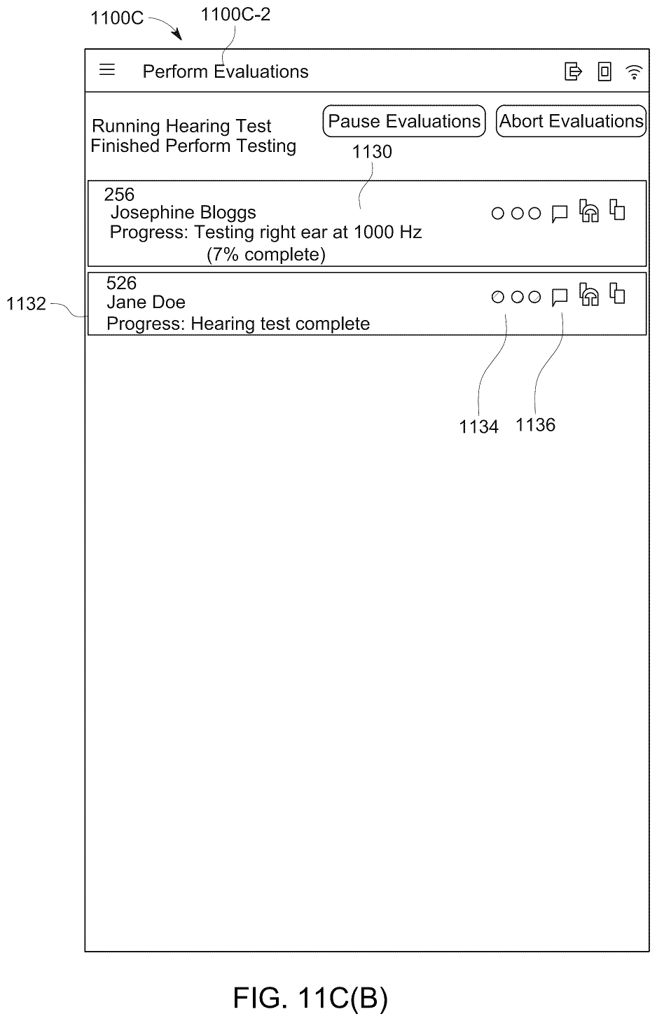

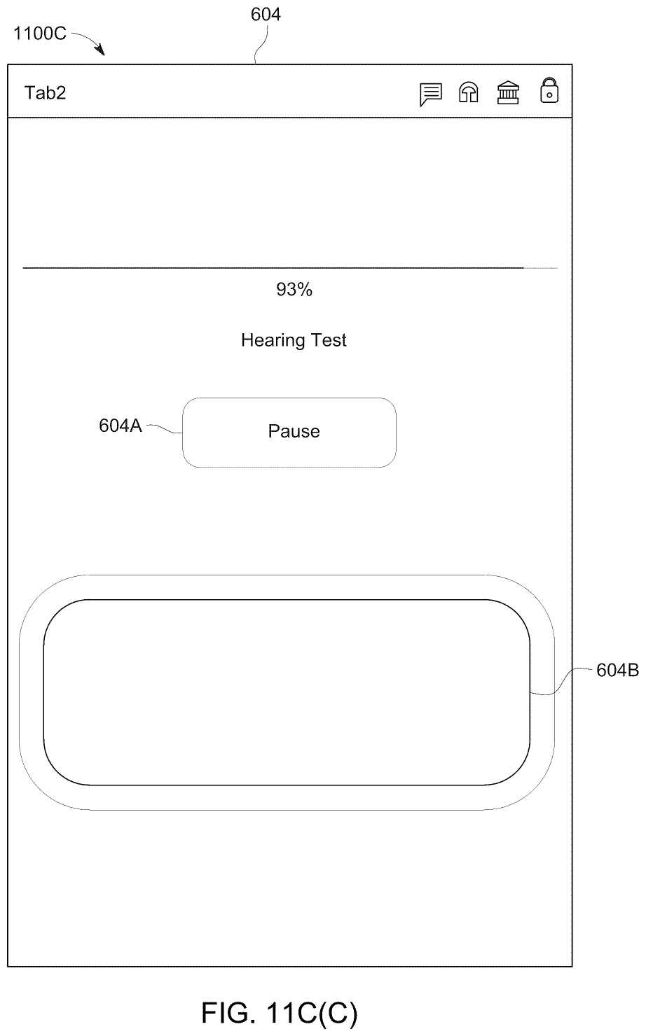

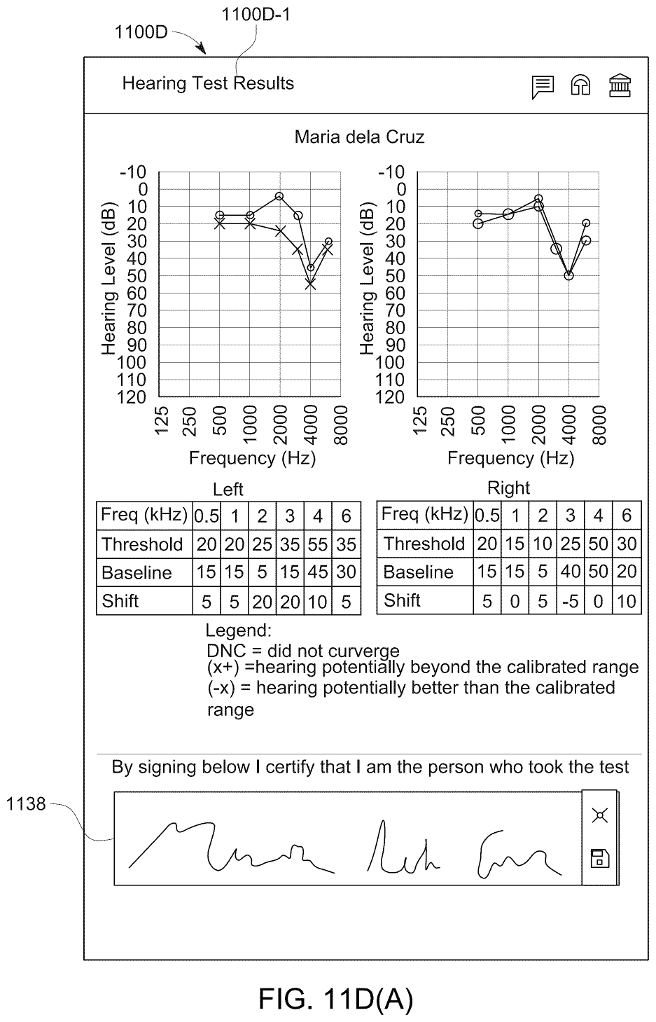

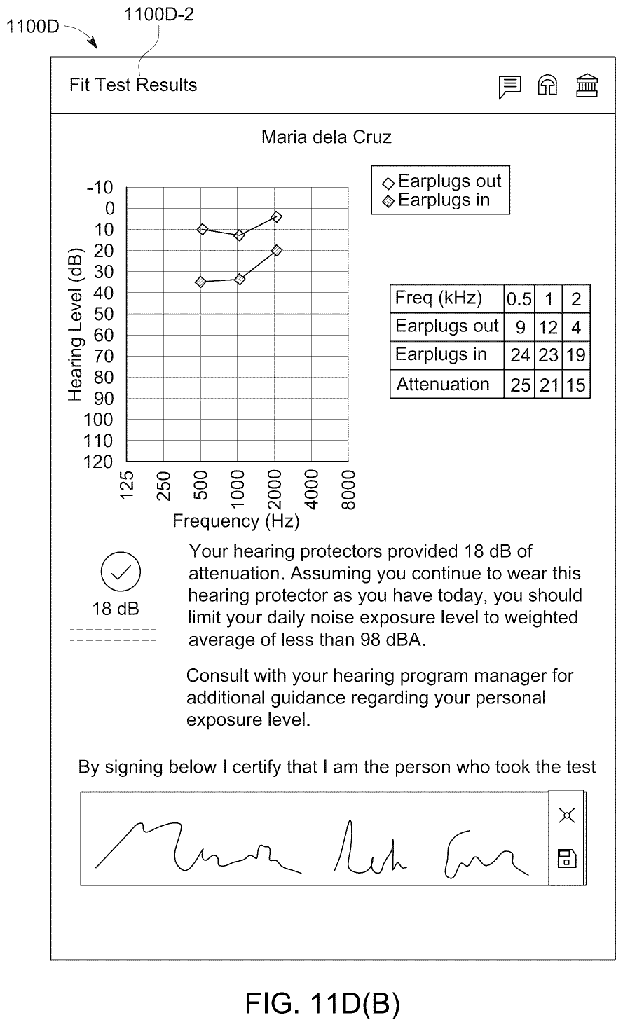

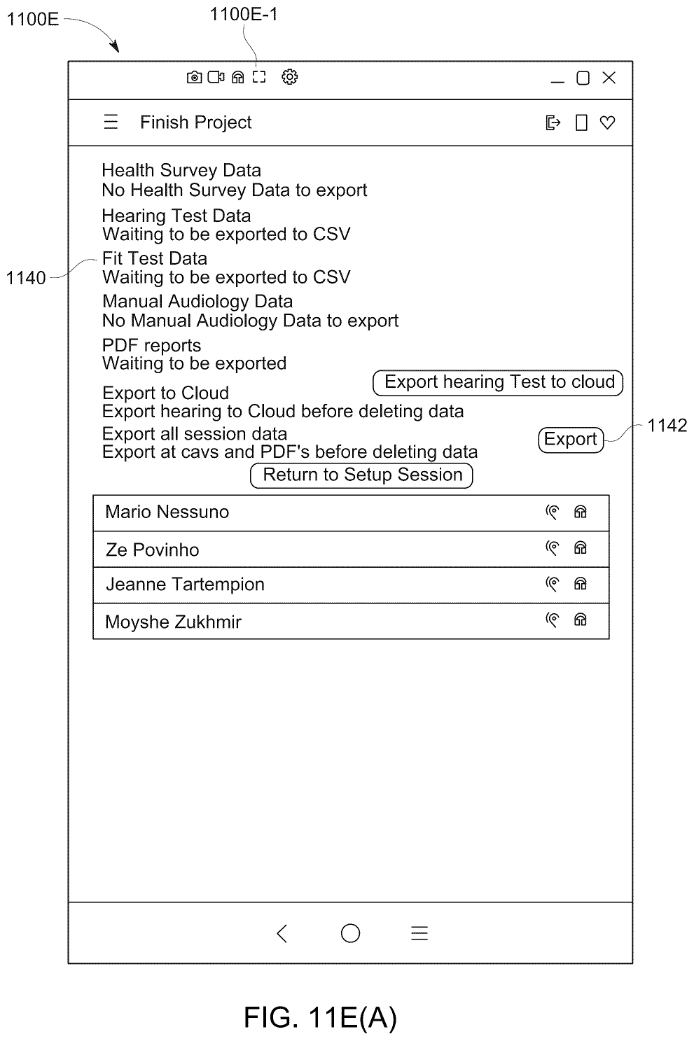

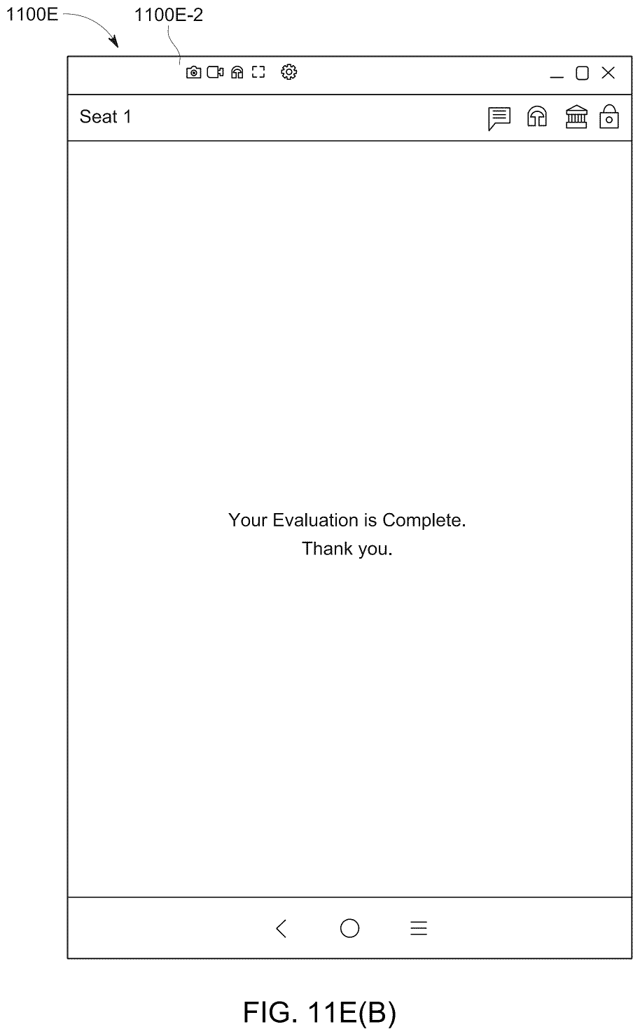

In operation O308, the administrator device 105 may process the connection status from the interface device 120 and perform certain actions. For instance, the administrator device 105 may launch the audiometer admin app 220B and display an administrator interface (e.g., the set up interface 1100A-1 of FIG. 11A) in response to the connection status. The administrator device 105 may receive user inputs (e.g., setting up test parameters) and a user input to a start test. The administrator device 105 may determine a first action based on the user input to start a test. For instance, the administrator device 105 may retrieve relevant data and generate a first instruction message. In some cases, the first instruction message does not include a signal that should be presented to the test taker. In some cases, the first instruction message does not include data indicating frequency of stimuli, amplitude of stimuli, or timing of stimuli.

In operation O310, the administrator device 105 may transmit the first instruction message. The first instruction message may include at least an indication of a test type to perform. In some cases, the first instruction message may include user data (e.g., for a welcome interface 1100B-2 on the interface device 120) and relevant audiometer data (e.g., previous test results, audiogram data for the user, unoccluded data for the user, etc., retrieved from the admin data structure 220C or the server 115).

In operation O312, the interface device 120 may process the first instruction message and perform certain actions. For instance, the interface device 120 may display an interface UI (e.g., the welcome interface 1100B-2, first instruction interface 602, and the like). The interface device 120 may receive various user inputs (e.g., confirming training, confirming data, etc.) and receive a user input to start the test. In some cases, the interface device 120 may transmit a message alerting the headset 125 that a test has been set up, but a user has not indicated that it should start via the interface device 120. In some cases, the interface device 120 may determine a second action based on the user input to start the test. For instance, the administrator device 105 may retrieve relevant data (e.g., from the first instruction message or the interface data structure 240C) and generate a second instruction message.

In operation O314, the interface device 120 may transmit the second instruction message to the headset 125. In some cases, the second instruction message may include at least an indication to start a test. In some cases, the second instruction message may also include a test type to perform. In some cases, the second instruction message may include user data (e.g., name, known medical conditions, etc.) and relevant audiometer data (e.g., previous test results, audiogram data for the user, unoccluded data for the user, etc., retrieved from the admin data structure 220C or the server 115 and passed in the first instruction message or by another message). In some cases, the test type to perform, the user data, and the audiometer data may be transmitted before the second instruction message (e.g., while the user is reviewing training material, etc.), such as in response to receiving the first instruction message. The headset 125 may receive the second instruction message.

In operation O316, the headset 125 may execute a watchdog function O316B and the interface device 120 may execute a watchdog function O316A, to confirm the second connection is still active and operable every set period of time, or if no message was transmitted or acknowledged by the other party within a threshold period of time. For instance, using Bluetooth, the headset 125 may broadcast an acknowledgement request message and the interface device 120 may receive, process, and transmit an acknowledge message to the headset 125. In this manner, the headset 125 may maintain safety of users by not increasing stimulus levels to high levels (e.g., upper range dB levels) while a user is trying to respond but the interface device 120 is not able to communicate the user's responses to the headset 125.

In operation O318, the headset 125 may process the second instruction message and any data included with the second instruction message, such as test type, user data, and/or audiometer data (collectively, “test parameter data”). In the case the test parameter data was sent before the second instruction message, the headset 125 may retrieve the test parameter data from memory onboard the headset 125 (where the test parameter data was stored when received). The headset 125 may proceed to determine a series of stimuli based on the test parameter data. The headset 125 may proceed to output a first stimulus at time t₁ , by, e.g., driving one or both speakers and/or a bone conductor. The headset 125 may also open a first response window.

In operation O320, the interface device 120 may receive a user input indicating a response via, e.g., the response UI element 604B. The interface device 120 may determine a third action based on the user response indicating a response to the stimulus. In some cases, the third action may be to transmit a response message to the headset 125. In some cases, the third action may be to transmit a response message to the administrator device 105 (so that the administrator device 105 may also collect response data). In this case, the headset 125 may periodically or in response to trigger conditions, transmit data regarding the output stimuli (e.g., time, frequency, level, and the like) to the administrator device 105, via the interface device 120.

In operation O322, the interface device 120 may transmit a response message to the headset 125. The headset 125 may receive the response message. The response message may indicate a timestamp of when the user input indicated a response via the response UI element 604B.

In operation O324, the headset 125 may determine the response was within the first response window. For instance, the headset 125 may extract the timestamp from the response message and compare it to the first response window. The headset 125 may record response data (e.g., response time (time elapsed from output to timestamp), user response, frequency, level, masking, ear tested, etc.). The headset 125 may then output a second stimulus at time t₂ . The headset 125 may also open a second response window.

In operation O326, the headset 125 may determine the second response window has ended. For instance, the response window may be set to a defined range (e.g., 3, 5, or 7 seconds). If the headset 125 may determine no response from the user within the second response window if no response message/timestamp of a response message is received from the interface device within the second response window. The headset 125 may process to record response data (e.g., no user response, frequency, level, masking, ear tested, etc.). The headset 125 and interface device 120 may continue the test by outputting stimuli (in accordance with the serious of stimuli) and passing response messages back and forth.

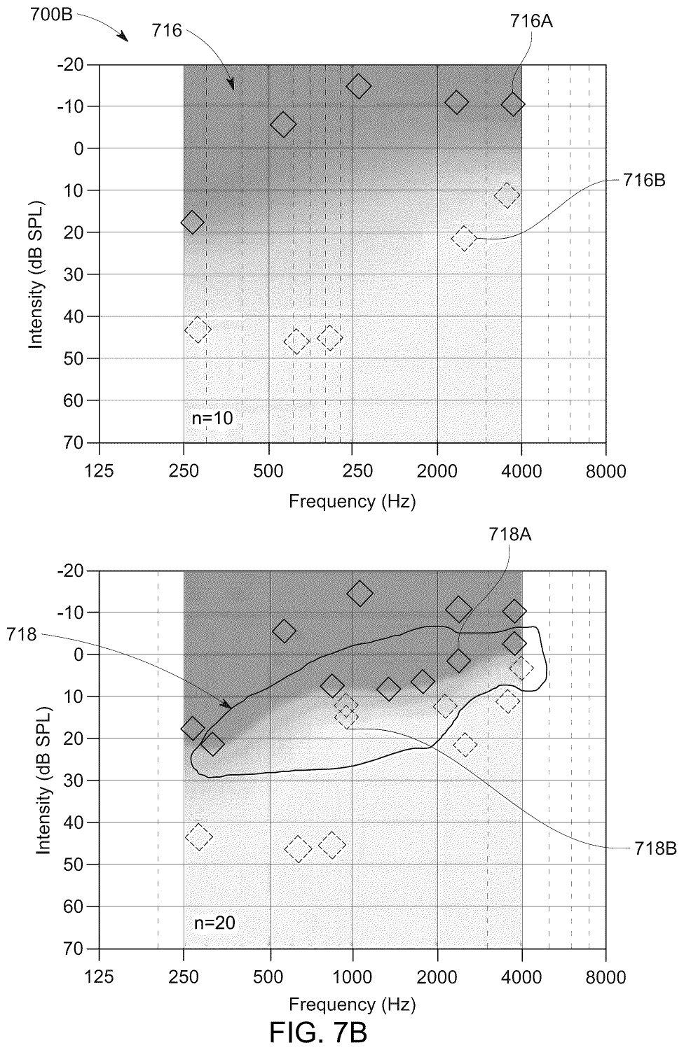

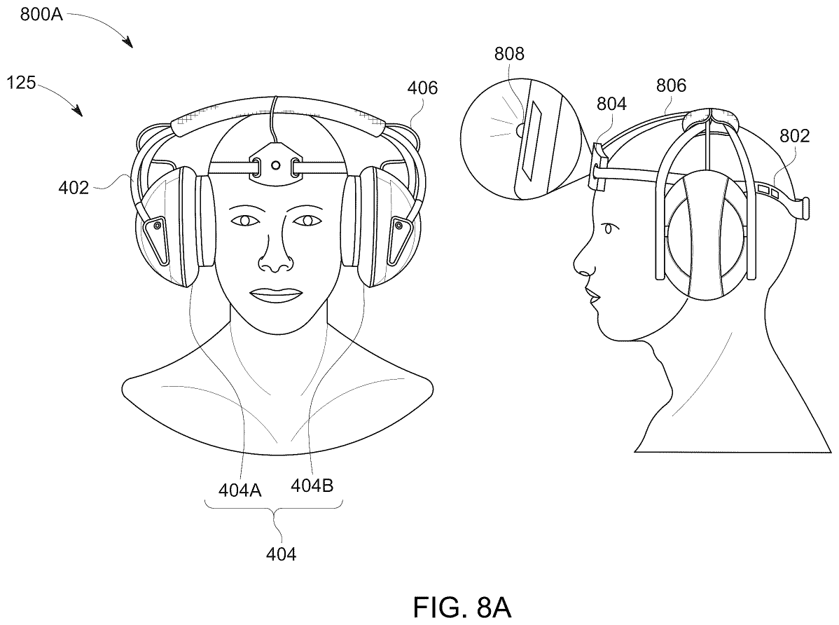

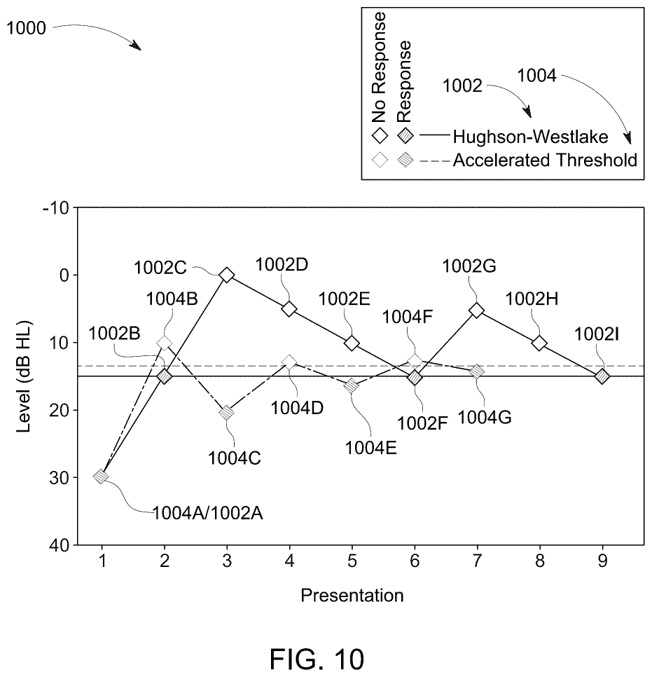

In operation O328, the headset 125 may determine to modify the series of stimuli. In some cases, the headset 125 may determine to modify the series of stimuli after a preset number of stimuli have been output or based on user's responses (or lack thereof). For instance, the headset 125 may determine that a change to the series may accelerate determination of a hearing threshold of a user faster by varying level or frequency in a certain manner (see FIG. 10), or by selecting an uncertain point on a threshold map 708 (see FIG. 7A). The headset 125 may then output a stimulus in accordance with the modified series of stimuli.

In some cases, response times may be used to modify the series of stimuli. For instance, longer response times may indicate guessing or stimuli that are close to a hearing threshold of the user. Generally, response times of users are generally consistent (e.g., one hundred ms). Thus, a response time near the average response (“valid response”) may indicate valid responses by the user. However, response times that exceed a threshold response time (such as ranging from 1, 2, or 3 seconds but are within the response window) more than the average response (“invalid responses”) may indicate guessing or uncertainty. In some cases, the headset 125 may modify the series of stimuli to re-test invalid responses. In some cases, the headset 125 may determine hearing thresholds using weighted thresholds. The weights for invalid responses may be weighted less than weights for valid responses. In some cases, the headset 125 may mark the response as invalid and omit the response from consideration. In some cases, the headset 125 may flag the user for intervention by the administrator.

In operations O330, O322, O324, the headset 125 and interface device 120 may operate as in operations O320, O322, and O324 (user response within time window). However, in addition, in operation O334, the headset 125 may also determine an intermediate result. The intermediate result may include a response dataset indicating what and when stimuli were output, whether responses were received, the response time for response, and the like for a portion of test.

In operation O336, the headset 125 may transmit the intermediate result to the interface device 120. The interface device 120 may receive the intermediate result from the headset 125.

In operations O338 and O340, the interface device 120 may process the intermediate result (e.g., store it locally) and/or transmit the intermediate result to the administrator device 105. The administrator device 105 may receive the intermediate result.

In operation O342, the administrator device 105 may process the intermediate result and update an administrator interface indicating progress and/or aspects of the response dataset. For instance, the administrator device 105 may update the second evaluation interface 1100C-2 to reflect the status and/or data of the response dataset.

The headset 125 and the interface device 120 may continue the test by outputting stimuli (in accordance with the modified serious of stimuli) and passing response messages back and forth. For instance, the headset 125 and interface device 120 may continue the test in parallel to the intermediate result being passed to the interface device 120 and/or the administrator device 105.

In operation O344, the headset 125 may determine the test is complete. For instance, for a fit test, the headset 125 may determine (statistically) the attenuation is sufficient for a hearing protector worn by the user. For a hearing test, the headset 125 may determine hearing thresholds for a set of frequencies and determine no more stimuli are required to determine the thresholds. The headset 125 may proceed to determine a final result. The final result may include a response dataset indicating what and when stimuli were output, whether responses were received, the response time for response, and the like for the entire test (or portions that have not been sent by intermediate results).

In operation O346, the headset 125 may transmit the final result to the interface device 120. The interface device 120 may receive the final result from the headset 125.

In operations O348 and O350, the interface device 120 may process the final result (e.g., store it locally) and/or transmit the final result to the administrator device 105. The administrator device 105 may receive the final result.

In operation O352, the administrator device 105 may process the final result and update an administrator interface indicating progress (e.g., test complete) and/or aspects of the response dataset. For instance, the administrator device 105 may update the second evaluation interface 1100C-2 to reflect the status and/or data of the response dataset, or display the report interface 1100E-1.

In FIG. 3E, the operations O354 through O370 are exemplary of how errors are handled. For clarity, these operations are optional with the above operations.

In operation O354, the headset 125 may determine the second connection was interrupted. For instance, the watchdog 416B may determine no response message or requested acknowledgement has been received in a time frame. The headset 125 may halt the series of stimuli until the second connection re-established.

In operations O356, the interface device 120 may determine the second connection was interrupted. For instance, the watchdog 416A may determine no result message or requested acknowledgement has been received in a time frame. In operation O358, the interface device 120 may proceed to transmit an error indicator to the administrator device 105. The error indicator may indicate an interrupted second connection. The administrator device 105 may receive the error indicator from the interface device 120. In operation O360, the administrator device 105 may process the error indicator and update the administrator interface (e.g., by alerting the administrator that an error has occurred).

In operation O362, the headset 125 may determine a startup error or runtime error has occurred. For instance, the headset 125 may determine masking is not feasible, and the like, for this user. The headset 125 may proceed to not start or halt the series of stimuli until the startup error or runtime error is resolved.

In operations O364, the headset 125 may transmit an error indicator to the interface device 120. The interface device 120 may receive the error indicator.

In operations O366 and O368, the interface device 120 may process the error indicator and proceed to transmit the error indicator to the administrator device 105. The administrator device 105 may receive the error indicator from the interface device 120. In operation O370, the administrator device 105 may process the error indicator and update the administrator interface (e.g., by alerting the administrator that an error has occurred).

In this manner, hearing testing may be automated while outside of a sound booth.

### Headset

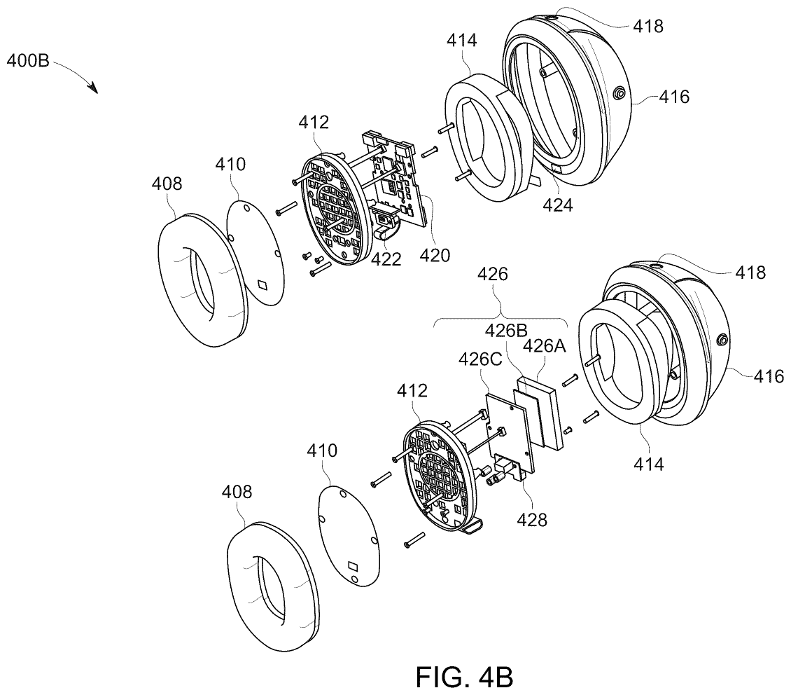

FIGS. 4A-4B depict features of a headset 125. The features of the headset 125 of FIGS. 4A-4B may apply to FIGS. 1, 2A-2B, 3A-3E, 5A-5C, 6A-6B, 7A-7B, 8A-8C, 9, 10, 11A-11E, 12, and 13.

FIG. 4A depicts external features of the headset 125. The headset 125 may include a support structure 402, a pair of ear cups 404 (including a first earcup 404A and a second earcup 404B), and a cable 406 connecting the pair of ear cups 404.

FIG. 4B. depicts internal features of the headset 125. In some cases, each of the pair of ear cups 404 of the headset 125 may include a cushion 408, a cover 410, an audiometer stack (which may be the same or different between the ear cups 404, but both always include a speaker assembly 412), insulation 414, a case 416, and a cable connection 418. In some cases one or both of the ear cups 404 of the headset 125 may include: a programmed board 420, a charging port 422, a charging hole 424, a battery system 426, and a switch board 428.

In some cases, an audiometer stack may include one of the programmed board 420 and/or the battery system 426. In some cases, the interface device 120 is configured to wirelessly connect to at least a first audiometer stack of the pair of ear cups. For instance, the interface device 120 may be configured to connect to the audiometer stack (“a first audiometer stack”) that has a programmed board 420. The second audiometer stack may include a battery system 426 configured to power each of the first and second audiometer stack.

In some cases, the first audiometer stack is connected to a second audiometer stack of the pair of ear cups 404 via the cable 406, so as to pass an analog signal to drive a speaker of the second audiometer stack from the programmed board 420. The cable 406 may also pass power from the second audiometer stack to the first audiometer stack. In some cases, the first audiometer stack includes control electronics and a wireless communication transceiver. For instance, the control electronics and a wireless communication transceiver may be hosted on the programmed board 420. In some cases, the wireless communication transceiver may be a Bluetooth drive. In some cases, the control electronics may be a controller and memory. In some cases, the control electronics are configured to receive, via the wireless communication transceiver, interface device instructions from the interface device 120. For instance, the interface device instructions may instruct the control electronics to start a test. The control electronics may in accordance with test parameter data and firmware of the control electronics, determine a series of stimuli. The control electronics may then, in accordance with the series of stimuli, output analog signals. The control electronics may transmit the analog signals to each speaker of the first and second audiometer stack. In some cases, the cable 406 may be omitted. In these cases, the first and second audiometer stack may include all or some of the components of the first audiometer stack (e.g., programmed board, battery, wireless communication transceiver, and analog outputs) to drive respective speakers and receive analog data from respective microphones. In some cases, the first and second audiometer stack may each receive instructions from the interface device 120, and coordinate stimuli output between themselves. In some cases, the first and second audiometer stack may omit the battery system. In these cases, the headset 125 may be powered by an external battery (e.g., a mobile battery plugged into the headsets) or by an external power supply (e.g., plugged into a local electrical grid).

In some cases, the control electronics are configured to transmit, via the wireless communication transceiver, data to the interface device 120 from each of the first and second audiometer stack. In some cases, the data may be based on microphone data from each of the first and second audiometer stack. In some cases, the data may only be transmitted from the programmed board 420 (e.g., intermediate results or final results). In some cases, the microphone of an audiometer stack may collect ambient noise data and/or calibration feedback noise data. In the case of ambient noise data, the headset 125 may during a hearing test, use the microphone to detect ambient noise and determine if the ambient noise is greater than an ambient noise threshold (e.g., a specific level of dB). In this case, the headset 125 may determine to pause the hearing test, transmit an alert (e.g., a runtime error indicator) to the interface device 120 and/or the administrator device 105. In some cases, alternatively or in addition to the headset 125 checking ambient noise, the interface device 120 and/or the administrator device 105 (if locally present) may use their own microphones to record ambient noise and determine if an ambient noise threshold is exceeded. The headset 125, the interface device 120, and/or the administrator device 105 may detect the ambient noise is below the ambient noise threshold, and determine to continue the test. In some cases, the interface device 120 may display an interface to the user to inform them that the test is paused due to ambient noise. In the case of calibration noise data, the headset 125 may, while performing a calibration check (see, e.g., calibration check interface 1100A-3 of FIG. 11A), output stimuli via speakers in an enclosed space (e.g., a cover is covering cushions), obtain calibration noise data (e.g., a microphone signal converted to digital data), and determine if the output stimuli satisfies a calibration condition (e.g., within a range of expected sound outputs from the speakers).

The cushion 408 may formed of insulation (e.g., for noise reduction) and covered in a fabric (e.g., for comfort). The cushion 408 may surround the ear of the user.

The cover 410 may be a dust cover for the speaker assembly 412. The speaker assembly 412 may include at least a speaker and a structure to mount the speaker inside the ear cup. In some cases, the speaker assembly 412 may also include a microphone. In some cases, the structure to mount the speaker inside the ear cup of the speaker assembly 412 may be a plate. In some cases, the speaker assembly 412 may also include additional elements to improve the acoustics (e.g., closed/open-cell foam), increase robustness to handling (e.g., fabric covers and rigid structures), and to measure additional data relevant to conducting hearing testing and fit testing (e.g., temperature, humidity, and pressure sensors).

The insulation 414 and the case 416 collectively may be “an ear cup attenuating structure.” The insulation 414 may be configured to attenuate high frequency noise from the exterior of the ear cup. The case 416 may be configured to attenuate low frequency noise from the exterior of the ear cup. The case 416 may include protruding baffles from an interior wall of the case towards the interior of the ear cup. The case 416 may include a side wall extending away from the cushion 408 and an end wall to enclose the side wall. In some cases, the end wall may be curved in at least one dimension to reduce resonance with the stimuli from a speaker of the audiometer stack. For instance, the end wall may be semi-circular with a radius extending from an ear canal, and the semi-circular extends in a vertical direction from a top of a user's head to a bottom of the user's head. In some cases, the case 416 may omit the protruding baffles from an interior wall of the case towards the interior of the ear cup. The cable connection 418 may pass through the case 416, to connect the cable 406.

In some cases, the programmed board 420 may include firmware, a voltage module, and at least one analog output. The firmware may functionally correspond to the control electronics or the controller and memory discussed above. The firmware may be configured to determine frequency of stimuli, amplitude of stimuli (e.g., level in dB), and timing of stimuli (collectively, test parameters) based on a type of hearing or fit test being performed. For instance, the test parameters correspond to a series of stimuli in accordance with test parameter data. In some cases, the firmware may adjust the test parameters based on user response and/or response time.

In some cases, the voltage module may be configured to output a dynamic range for each of the at least one analog output. An output of the dynamic range may be configured to produce a pure tone, warble tone (e.g., frequency modulated tone), bandlimited noise, speech, or other stimulus designed to test the fit of an earplug or hearing sensitivity of the patient.

The charging port 422 may be a USB port to receive power from the charging hole 424 (when USB power applied to the charging hole 424. The charging hole 424 may be formed in the case 416.

The battery system 426 may include a battery 426A, a battery attachment 426B, and a battery plate 426C. The battery 426A may be a rechargeable battery. The battery attachment 426B may secure the battery 426A to the battery plate 426C. The battery plate 426C may secure the battery system 426 to the speaker plate assembly and reduce vibration/noise.

The switch board 428 may route analog signals, via the cable 406, between the microphone/speaker of the second audiometer stack and the first audiometer stack. The switch board 428 may also route battery power from the battery system 426, via the cable 406, from the second audiometer stack to the first audiometer stack.

### Fit Testing

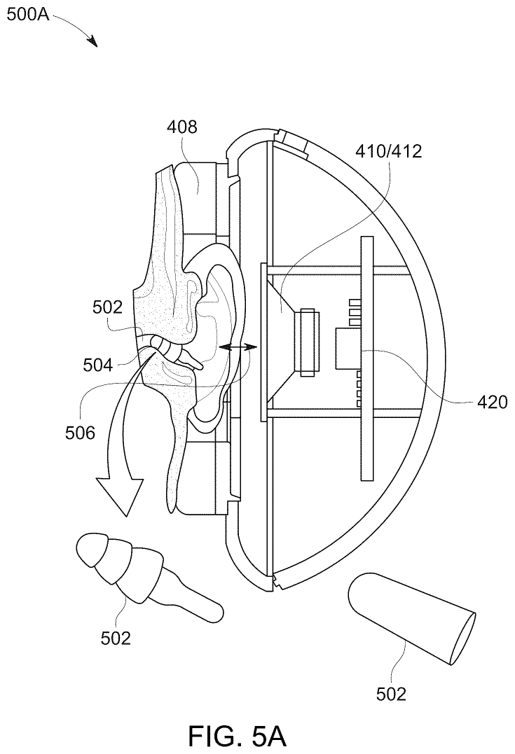

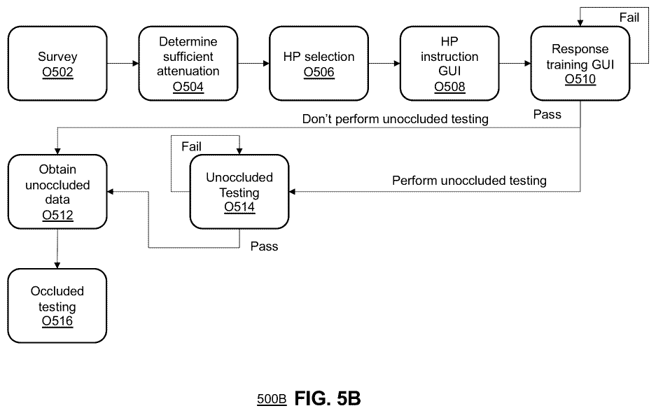

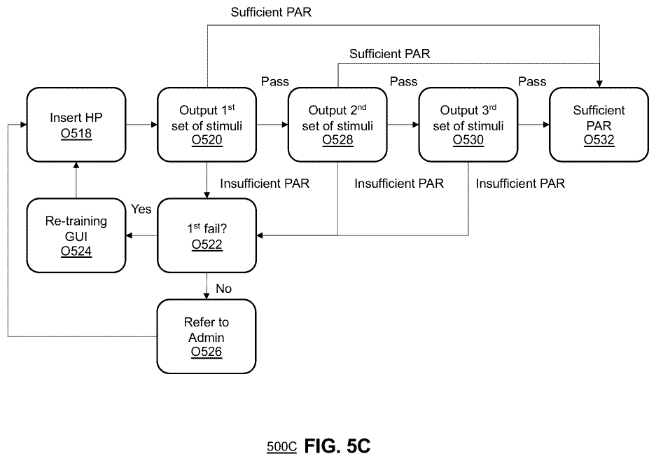

FIGS. 5A-5C, 6A-6B, and 7A-7B depict features of fit testing. The features of fit testing of FIGS. 5A-5B, 6A-6B, and 7A-7B may apply to FIGS. 1, 2A-2B, 3A-3E, 4A-4B, 8A-8C, 9, 10, 11A-11E, 12, and 13.

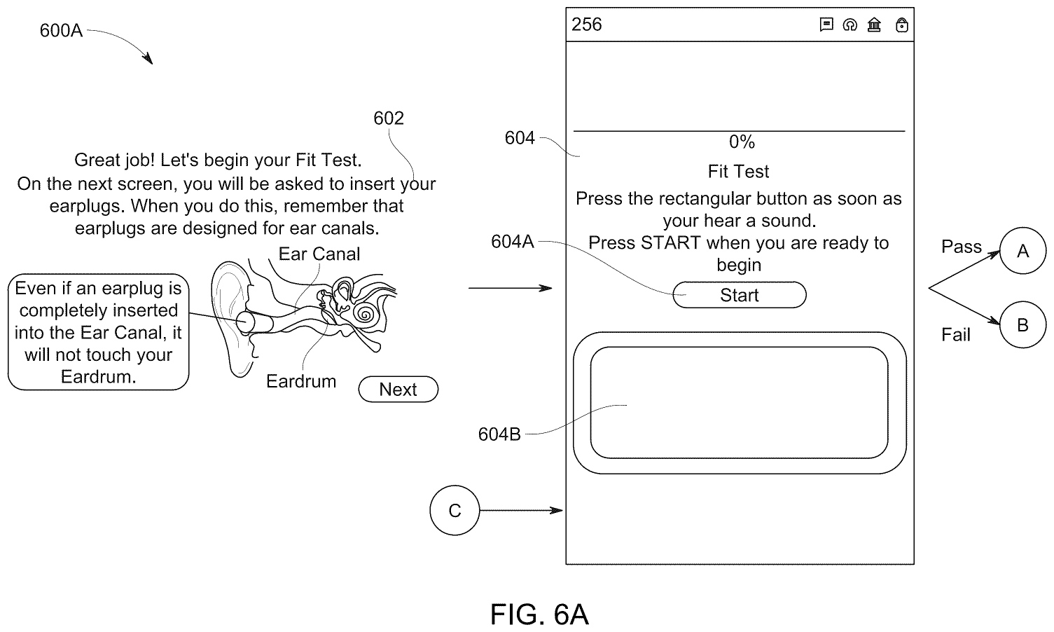

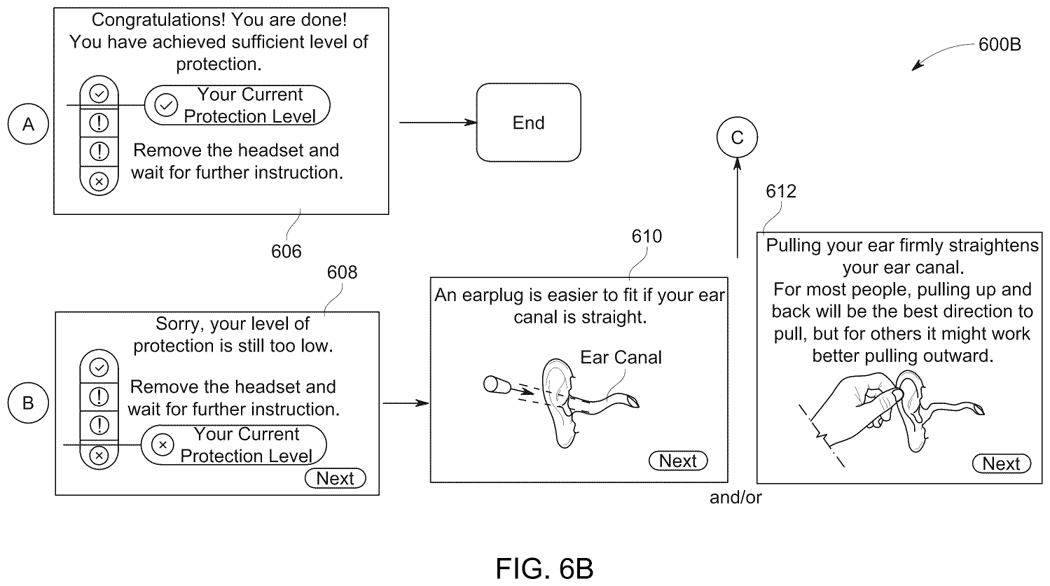

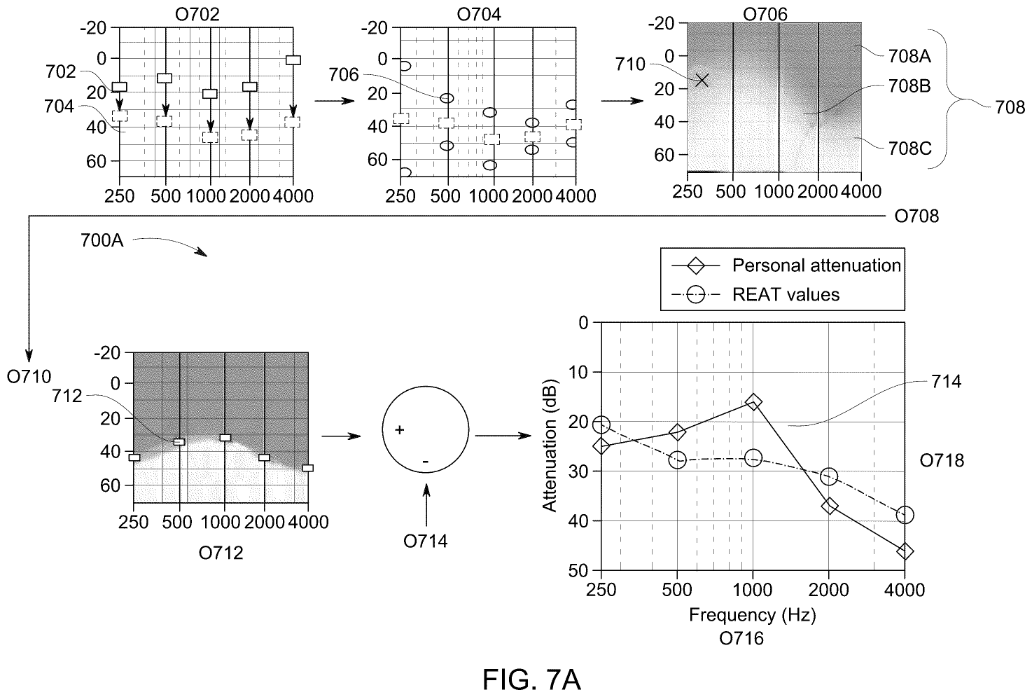

FIG. 5A depicts a fit test diagram 500A for fit testing. FIGS. 5B-5C depict flowcharts 500B and 500C for fit testing. FIGS. 6A-6B depict diagrams 600A and 600B for graphic user interface sequences for fit testing. FIGS. 7A-7B depict a dataflow 700A to determine an attenuation rating (e.g., a personal attenuation rating, “PAR”) and comparison charts 700B data based on results of the dataflow 700A.

In FIG. 5A, the fit test diagram 500A may depict an ear canal 502 and a hearing protector 504 inserted in the ear canal 502 with respect to the cushion 408, the cover 410, the speaker assembly 412, and, e.g., the programmed board 420 (or the battery system 426/switch board 428). The cover 410 and the speaker assembly 412 may be positioned with a clearance 506 from an ear of the user. The clearance 506 may be defined so that a plate of the speaker assembly 412 is position inside the interior of the ear cup so as to provide a space for hearing protectors when applied to the user. For instance, the space of the clearance 506 may be 20, 30, 40 mm, and the like. In some cases, the mounting structure (e.g., a plate) is configured to mount the speakers inside the earcups. The speakers and mounting structure are positioned inside the ear cup with sufficient clearance to avoid physical contact with the test taker's ears and any inserted hearing protectors. In this manner, the clearance avoids physical contact between (a) the speakers and mounting structure, and (2) the ear or the hearing protector. Thus, the clearance can avoid alternate pathways for sound/vibrations, which could lead to incorrect results.

Generally, the system may be configured to output a first interface. The first interface may include a first interface element configured to be selectable by a first input to start a hearing test. The system (e.g., interface device 120 or administrator device 105) may determine that a first input has selected the first interface element. In response to determining the first input has selected the first interface element, the system (e.g., the interface device 120) may instruct at least the first audiometer stack to perform a first series of stimuli. For instance, the programmed board 420 of the headset 125 may determine the first series of stimuli in accordance with test parameter data for a hearing test, and drive speakers and/or a bone conductor to output the first series of stimuli. The system may proceed to receive a first set of one or more user responses for at least a first subset of the first series of stimuli. For instance, user inputs on the interface device 120 may indicate user responses to stimuli. Based on the first set of one or more user responses, the system may determine a hearing test result of the hearing test. In some cases, the hearing test result may include at least a measure of a hearing ability of the user. For instance, the measure of hearing ability may correspond to unoccluded data for the user.

The system may proceed to output a second interface. The second interface may be configured to indicate a transition to a hearing protector fit test (“fit test”). The second interface may include at least a second interface element configured to be selectable by a second input to start the hearing protector fit test. The system may proceed to determine a second input has selected the second interface element, and, in response to determining the second input has selected the second interface element, instruct at least the first audiometer stack to perform a second series of stimuli. For instance, the programmed board 420 of the headset 125 may determine the second series of stimuli in accordance with test parameter data for fit testing, and drive speakers and/or a bone conductor to output the second series of stimuli. The system may proceed to receive a second set of one or more user responses for at least a second subset of the second series of stimuli. Based on the second set of one or more user responses, the system may determine a fit test result of the fit test. In some cases, the fit test result may include an indication of a hearing attenuation achieved by a hearing protector as worn by the user during the fit test.

In some cases, the system may determine a portion of the first series of stimuli based on a portion of the first set of one or more user responses. For instance, the system may modify the modify the series of stimuli after a preset number of stimuli have been output or based on user's responses (or lack thereof). For instance, the headset 125 may determine that a change to the series may accelerate determination of a hearing threshold of a user faster by varying level or frequency in a certain manner (see FIG. 10), or by selecting an uncertain point on a threshold map 708 (see FIG. 7A).

In FIGS. 5B-5C, the flowcharts 500B and 500C may depict operations in response to an instruction message from the administrator device 105, via the interface device 120, to start and conduct a fit test. The operations may be performed by the headset 125 and the interface device 120.

In operation O502, the system 125 may include a survey. The survey may include questions to determine noise exposure, such as asking about their occupation, or in the case of military, their military occupational specialty (MOS), and may include ability to input data from noise measurements made in the environments they work, and the like. The survey may be digital (e.g., via the interface device 120) or data via forms and input (e.g., by an administrator).

In operation O504, the headset 125 may determine sufficient attenuation. For instance, the headset 125 may determine the sufficient attenuation based on their expected noise exposure as determined from the survey.

In operation O506, the interface device 120 may receive a hearing protector (HP) selection. For instance, the UI 225 may display different types of hearing protector and the user may make a selection from the different types.

In operation O508, the interface device 120 may display hearing protection training interfaces. For instance, the hearing protection training interfaces may correspond to the selected hearing protector and/or include general hearing protector information (e.g., about the ear canal).

In operation O510, the headset 125 and interface device 120 may perform response training by displaying a response interface on the interface device 120 and/or outputting high confidence stimuli for the user to respond. The headset 125 and the interface device 120 may determine whether the user has passed or failed the response training (e.g., no response or inconsistent responses). If the user fails the response training, the system may re-attempt training. If the user fails again, the system may re-route the user to assistance with the administrator.

In some cases the headset 125 and interface device 120 do not need to perform unoccluded testing to obtain unoccluded data for the user (e.g., already have unoccluded data for the user or results from a hearing test). In this case, in operation O512, the headset 125 may use these earlier results for estimating the unoccluded data for the user.

In some cases the headset 125 and interface device 120 do need to perform unoccluded testing to obtain unoccluded data for the user (e.g., do not already have non-stale unoccluded data for the user). In this case, in operation O514, the headset 125 and the interface device 120 may perform unoccluded testing to obtain the unoccluded data for the user. For instance, the system may perform a hearing test to determine the unoccluded data. If the user fails the unoccluded testing, the system may re-attempt the unoccluded testing. If the user fails again, the system may re-route the user to assistance with the administrator.

In operation O516, the headset 125 and the interface device 120 may perform occluded testing. Operations O518 through O532 may be exemplary operations to perform occluded testing.

In some cases, the headset 125 may before determining the fit test result of the fit test and while performing a second series of stimuli for the fit test: determine, for a user response responsive to a specific frequency and amplitude, that the hearing protector is improperly applied to the user. In response to determining the hearing protector is improperly applied to the user, the headset 125 may cause the interface device 120 to output (e.g., display) an instruction to adjust the hearing protector before continuing the hearing protector fit test, or to seek assistance from an administrator.

In operation O518, the interface device 120 may instruct the user to insert the hearing protector. In operation O520, the headset 125 may output a first set of stimuli. Based on user responses (or lack thereof), the headset 125 may determine the user passed the first set of stimuli, the user has sufficient PAR, or that the user has an insufficient PAR.

For instance, the first set of stimuli may be at a frequency of 500 Hz. As an example, assume the user needs to achieve a PAR of 18 dB. The headset 125 may determine, based on statistics, that if the user achieved an attenuation of 9 dB (or less) at 500 Hz, that the user would be unlikely to achieve a PAR of 18 dB. This poor performance would indicate the test should be halted for the user to try re-fitting the hearing protector or trying another model of hearing protector. Conversely, if the user achieved an attenuation of more than 25 dB, it is highly likely they have achieved a PAR of 18 dB and testing may be halted (i.e., proceed directly to sufficient PAR O532). If the user achieves an attenuation in between these two extremes, testing at additional frequencies may be required to determine if they have met the required PAR.

In operation O528, in response to passing the first set of stimuli, the headset 125 may output a second set of stimuli. Based on user responses (or lack thereof), the headset 125 may determine the user passed the second set of stimuli, the user has sufficient PAR, or that the user has an insufficient PAR.

For instance, the second set of stimuli may be at a frequency of 1000 Hz. The headset 125 may determine that the user has an insufficient PAR if an A-weighted attenuation for 0.5 and 1.0 kHz is less than 12 dB. Conversely, if the user achieved an attenuation of more than 25 dB, it is highly likely they have achieved a PAR of 18 dB and testing may be halted (i.e., proceed directly to sufficient PAR O532). For instance, based on statistics, the user is unlikely to achieve a sufficient PAR of 18 dB.

In operation O530, in response to passing the second set of stimuli, the headset 125 may output a third set of stimuli. Based on user responses (or lack thereof), the headset 125 may determine the user passed the third set of stimuli or that the user has an insufficient PAR. In operation O532, in response to passing the third set of stimuli, the headset 125 may determine the user has a sufficient PAR.

For instance, the third set of stimuli may be at a frequency of 2000 Hz. The headset 125 may determine that the user has an insufficient PAR if an A-weighted attenuation for 0.5, 1.0, and 2.0 kHz is less than 19 dB. For instance, based on statistics, the user is unlikely to achieve a sufficient PAR of 18 dB.

In operation O522, in response to the user having an insufficient PAR (from O520, O528, or O530), the headset 125 may determine whether this is the first fail of the user for occluded testing.

In operation O524, in response to determining this is the first fail of the user for occluded testing, the system may display a re-training interface to assist the user with hearing protectors and/or testing. The system may then proceed to re-attempt occluded testing at operation 518.

In operation O526, in response to determining this is not the first fail of the user for occluded testing (e.g., second or more fails), the system may refer the user to an administrator for assistance. The system may then proceed to re-attempt occluded testing at operation 518.

In FIGS. 6A-6B, the diagrams 600A and 600B may depict graphical user interfaces 602, 604, 606, 608, 610, and 612 to guide a user before, during, and after a fit test. The diagrams 600A and 600B may start at a first instruction interface 602. The first instruction interface 602 may include graphics and/or instructions on how to wear hearing protectors.

The interface device 120 may proceed to display a test interface 604. The interface device 120 may proceed to display the test interface 604 in response to a user input indicating the user is ready to proceed with the test. The test interface 604 may include a start/pause UI element 604A and a response UI element 604B. The start/pause UI element 604A may be user selectable to start/pause the test sequence. The response UI element 604B may be user selectable to indicate the user sensed the stimulus output by the headset 125. The interface device 120 may in response to a user input on either the start/pause UI element 604A or the response UI element 604B, transmit a message to the headset 125. The headset 125 may start/pause the test in response to the message indicating a start/pause. The headset 125 may record the response in response to the message indicating a response to the stimulus. The headset 125 and the interface device 120 may cooperate to complete the fit test by the headset 125 outputting stimuli (e.g., sound outputs for air conduction testing or vibration outputs for bone conduction testing) and the interface device 120 transmitting responses based on user interaction with the response UI element 604B to the headset 125. After the fit test is completed, the headset 125 may transmit a final result message to the interface device 120. The final result message may indicate the fit test was passed (e.g., sufficient attenuation) or failed (e.g., insufficient attenuation).

The interface device 120 may proceed to display a pass interface 606 (sequence A) or a failure interface 608 (sequence B), in accordance with the indication included in the final result message from the headset 125. The pass interface 606 may display graphics and/or data regarding the fit test, such as a level of attenuation a hearing protector provides the user. In the case of sequence A, the interface device 120 may proceed to end the fit testing.

The failure interface 608 may display graphics and/or data regarding the fit test, such as a level of attenuation (or lack thereof) a hearing protector provides the user. In the case of sequence B, the interface device 120 may proceed to display a first instruction interface 610 and/or a second instruction interface 612. The first instruction interface 610 and/or the second instruction interface 612 may include different graphics and/or instructions on how to wear hearing protectors. The interface device 120 may proceed to point C. Point C may redirect the user to re-try the fit test by displaying the test interface 604 and re-conducting the fit test.

In FIG. 7A, the dataflow 700A may determine a PAR for a user by performing a sequence of operations.

In operation O702, the headset 125 may predict occluded thresholds 704. For instance, the headset 125 may predict the occluded thresholds 704 based on a user's audiogram 702 and real-ear attenuation at threshold (“REAT”) values of a hearing protector selected for the fit testing.

In operation O704, the headset 125 (in conjunction with the interface device 120) may test a first series of stimuli 706 (e.g., a sound output for air conduction or vibration output for bone conduction). The first series of stimuli 706 may be selected (e.g., by pseudorandom selection) above and below the predicted occluded thresholds 704. The headset 125 may obtain (via the interface device 120) responses (or not) for none, one, or a plurality of the first series of stimuli 706. The headset 125 may determine a response dataset based on the responses (or not) and the first series of stimuli 706.

In operation O706, the headset 125 may determine a threshold map 708. For instance, the headset 125 may determine a likelihood map using a Gaussian process and the response dataset. The Gaussian process may determine a plurality of likelihoods based on the response dataset. Each of the plurality of likelihoods corresponding to a point in a frequency and level space (e.g., stimulus level in dB for a sound output for air conduction or vibration output for bone conduction) above or below the user's occluded threshold. The threshold map 708 may include a first region 708A, a second region 708B, and a third region 708C. The first region 708A may correspond to a high confidence likelihood (e.g., above a threshold confidence) that the frequency/level is below the user's threshold of hearing. The second region 708B may correspond to an uncertain region (e.g., the confidence likelihood is below the threshold confidence). In this case, the headset 125 may determine insufficient data is available to form a high confidence likelihood. The third region 708C may correspond to a high confidence likelihood (e.g., above the threshold confidence) that the frequency/level is above the user's threshold of hearing.

In operation O708, the headset 125 may test a second series of stimuli and re-determine the threshold map 708. For instance, the headset 125 may select a point 710 (frequency and level) with a highest uncertainty as a next stimulus (e.g., a sound output in air conduction or vibration output in bone conduction) to test. In some cases, the headset 125 may iteratively, select a point 710, test, and re-determine the threshold map 708 based on the updated response dataset, before testing a next point. In some cases, the headset 125 may select more than one point (e.g., a plurality of points) to test, test the more than one point, and re-determine the threshold map 708 based on the updated response dataset. In this manner, the fit test may reduce test time by testing highest uncertainty points, thereby reducing the second region in an efficient manner. In some cases, the fit test process may reduce test time by avoiding additional presentations to discover the occluded threshold of the user. For instance, the fit test may save two or more presentations (˜5 to 10 seconds, or more, of test time). While not a large time savings for a single test or user, over thousands or millions of tests the aggregate time saved may be substantial. The aggregate saved time may be especially useful for large organizations, such as manufacturing, military, or other high-noise risk occupations.

In operation O710, the headset 125 may determine a stopping condition is satisfied. For instance, the headset 125 may determine the stopping condition is satisfied when a pre-determined number of stimuli have been presented; the second region (as sampled across the domain of frequencies/levels for the second region) is less than a width threshold; or when the second region has an area less than a threshold area. The width threshold may be a predefined (or dynamic) range of stimulus levels (e.g., 5 dB, 4 dB, 3 dB, 2 dB, 1 dB, or 0.5 dB). For instance, the headset 125 may determine the distance between the first region and the third region at pre-selected frequencies (e.g., 250 Hz, 500 Hz, 1000 HZ, 2000 Hz, 4000 Hz and the like) are all below the width threshold.

In operation O712, the headset 125 may determine occluded data. The occluded data may include, e.g., occluded thresholds (e.g., discrete points) or a function (e.g., a curve or split function) describing a threshold boundary of hearing for a user when the user's ear canal is occluded. For instance, the headset 125 may determine occluded thresholds 712 by selecting a point with a lowest level (e.g., smallest dB) in the third region at the pre-selected frequencies.

In some cases, operations O702 through O712 may be used to determine unoccluded data. In this case, operation O702 may predict unoccluded thresholds 704 from prior tests or use default expected hearing range given a user's biographic data (e.g., age, occupation, etc.). Operations O704 through O712 may proceed in a similar manner, but in operation O712 the headset 125 may determine unoccluded data.

In operation O714, the headset 125 may obtain unoccluded data. The unoccluded data may include unoccluded thresholds (e.g., discrete points) or a function (e.g., a curve or split function) describing a threshold boundary of hearing for a user when the user's ear canal is unoccluded. The unoccluded data may be retrieved from prior testing (e.g., immediately before the fit test) or from historical data (e.g., from a last hearing or fit test that the user completed, within a threshold period of time).

In operation O716, the headset 125 may determine frequency attenuations for pre-selected frequencies 714. For instance, the headset 125 may determine the frequency attenuations for pre-selected frequencies by subtracting unoccluded data from the occluded data at each frequency of the pre-selected frequencies. For instance, the headset 125 may subtract unoccluded thresholds from the occluded thresholds (as discrete points or of the respective functions, or combinations thereof) at each frequency to determine a frequency attenuation for each frequency.

In some cases, the headset 125 may determine a series of stimuli (for fit testing) based on a first set of one or more user responses (from hearing testing). For instance, the first set of one or more user responses may determine unoccluded thresholds. The headset 125 may also determine the fit test result (e.g., PAR) based on the first set of one or more user responses (e.g., the unoccluded thresholds) and a second set of one or more user responses from the fit testing (e.g., the occluded thresholds).

In operation O718, the headset 125 may determine a PAR for the user based on the frequency attenuations for pre-selected frequencies 714. For instance, the headset 125 may determine the PAR by combining attenuations across frequencies (of the pre-selected frequencies) and expressed as a single value. The PAR may be used to assess if hearing protectors are providing adequate attenuation (e.g., a PAR value that satisfies a PAR condition, such as a threshold PAR value).

In FIG. 7B, the charts 700B may depict response datasets overtime. In the left chart, a first series of stimulus 716 are graphed at different points. Points 716A indicate no response and points 716B indicate a response. In the right chart, a second series of stimulus 718 are graphed at different points with the first series of stimulus 716. Points 718A indicate no response and points 718B indicate a response.

### Bone Conduction Testing

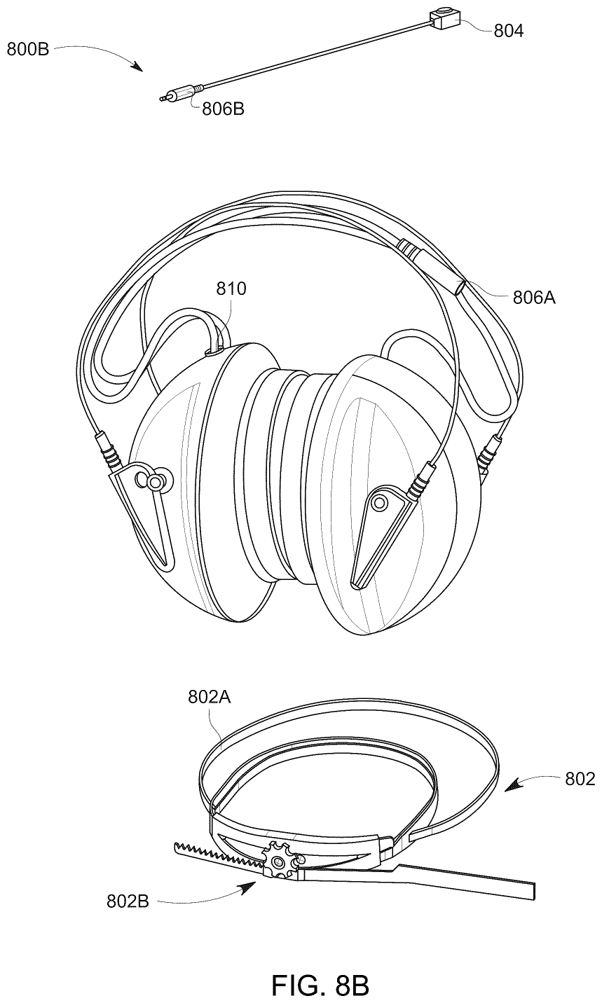

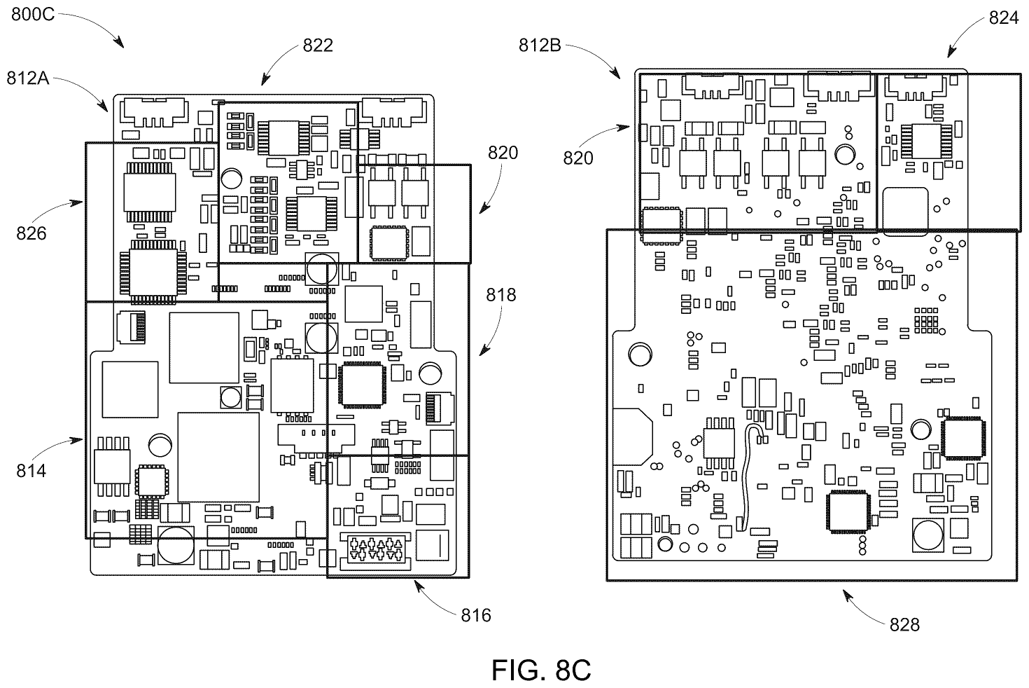

FIGS. 8A-8C depict features of bone conduction testing. The features of bone conduction testing of FIGS. 8A-8C may apply to FIGS. 1, 2A-2B, 3A-3E, 4A-4B, 5A-5C, 6A-6B, 7A-7B, 9, 10, 11A-11E, 12, and 13.

FIG. 8A depicts a diagram 800A of external features of a bone conducting headset 125. FIG. 8B depicts a diagram 800B of features of external components of the bone conducting headset 125. The bone conducting headset 125 includes the same components as depicted in FIG. 4A (e.g., the support structure 402, the pair of ear cups 404, and the cable 406), but also includes a bone conductor support structure 802, a bone conductor 804, a bone conductor cable 806. In some cases, the bone conducting headset 125 also includes a pressure monitor 808. Generally, the bone conductor 804 may be controlled (e.g., driven by analog signals) from the programmed board 800C (see FIG. 8C).

In some cases, the bone conductor cable 806 may be omitted. In these cases, the bone conductor 804 include all or some of the components of the first audiometer stack (e.g., programmed board, battery, wireless communication transceiver, and analog outputs) to drive the bone conductor 804 (while the first and second audiometer stack drive one or both respective speakers). In some cases, the bone conductor 804, and the first and second audiometer stack may each receive instructions from the interface device 120, and coordinate stimuli output between themselves.

FIG. 8C depicts a programmed board 800C of the bone conducting headset 125. The programmed board 800C may include a controller and memory 814, a power interface 816, a communication module 818, a voltage module 820, output filters 822, input filters 824, ADC and DAC converters 826, and a power circuit 828.

The bone conductor support structure 802 may include a headband 802A and an adjustment mechanism 802B. The adjustment mechanism 802B may include Velcro/elastic headband or a twist mechanism for tightening/loosening the headband 802A. The twist mechanism may cause gear teeth at one end of the headband 80A to ratchet tighter/looser. The gear teeth may be captured internally for safety and/or comfort. Thus, the bone conductor support structure 802 may be configured to hold the bone conductor 804 on the head of the user. The bone conductor support structure 802 may be configured to be adjustable to apply a variable fit and/or an amount of contact pressure between the bone conductor 804 and the head of the user.

The bone conductor 804 may couple (via the bone conductor support structure 802) to a head of a user. The bone conductor 804 may transmit physical vibrations to the head of the user (e.g., via a transducer). In cases where the bone conducting headset 125 includes the pressure monitor 808, the pressure monitor 808 may be positioned between the bone conductor 804 and the head of the user. In some cases, the pressure monitor 808 may be connected to a light or speaker to output a signal when a threshold contact pressure has been applied between the bone conductor and the head of the user. For instance, the threshold contact pressure may be sufficient for hearing/fit testing.

The bone conductor cable 806 may include a first part 806A and a second part 806B. The first part 806A may couple to the programmed board 800C (see FIG. 8C) via a hole 810 in the case 416. The second part 806B may couple to the first part 806A and the bone conductor 804, e.g., via audio jacks. In some cases, the bone conductor 804 is configured to be electronically connected to the first audiometer stack (where the programmed board 800C is located) of the pair of ear cups 404, via the bone conductor cable 806.

The controller and memory 814 may correspond to the firmware or control electronics discussed above. The controller and memory 814 may determine series of stimuli to be output by the speakers and/or the bone conductor. The power interface 816 may be connected to the charging hole 424 to route power to the battery system 426. The power circuit 828 may distribute power from the battery system 426.

The communication module 818 may include network I/O for Bluetooth communication. For instance, the communication module 818 may include a Bluetooth low energy module and a Bluetooth voice module.

The voltage module 820 may output a dynamic range for each of the at least two analog outputs. An output of the dynamic range may be configured to be a pure tone or more complex stimuli, such as narrow band noise, speech, etc.

The output filters 822 may receive analog signals from the DAC 826, manage signal quality, and output the filtered signal to the voltage module 820. The input filters 824 receive analog signals from the microphones, manage signal quality, and input the filtered analog signals to the ADC 826.

The DAC 826 may convert digital instructions (for stimuli) from the controller and memory 814 into output analog signals to be filtered by the output filters 822. The ADC 826 converts the filtered analog signals (from the microphones) to digital data.

In some cases, the first audiometer stack may include a first analog output, a second analog output, and a third analog output. The first analog output may drive a first speaker associated with the first audiometer stack. The second analog output may drive a second speaker associated with the second audiometer stack. The third analog output may drive the bone conductor.

In some cases, the third analog output may be different than the first analog output and the second analog output. In some cases, the first analog output, the second analog output, and the third analog output are each configured to output a respective dynamic range. In some cases, the first analog output, the second analog output, and the third analog output are configured to be driven separately. In some cases, the third analog output has a different dynamic range than the first analog output and the second analog output. In some cases, the dynamic range of the third analog output is smaller than the dynamic range of the first analog output and the second analog output. In some cases, the dynamic range of the third analog output has a lower upper range than the dynamic range of the first analog output and the second analog output.

### Masking During Testing

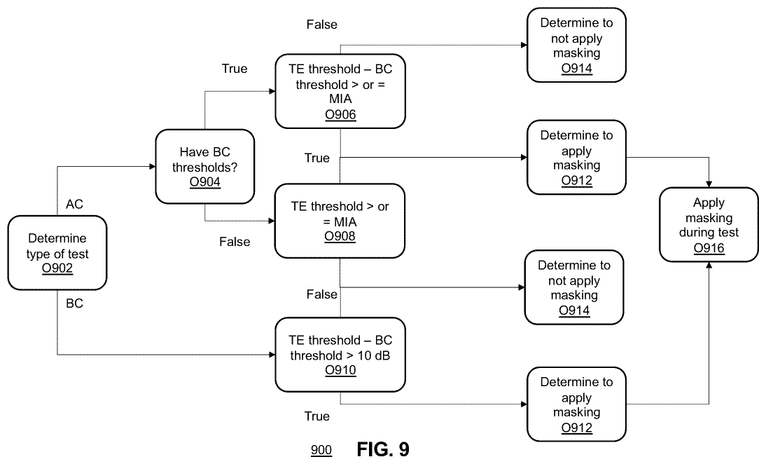

FIG. 9 depict a flowchart 900 for masking during hearing testing. The flowchart 900 for masking during hearing testing of FIG. 9 may apply to FIGS. 1, 2A-2B, 3A-3E, 4A-4B, 5A-5C, 6A-6B, 7A-7B, 8A-8C, 10, 11A-11E, 12, and 13. The flowchart 900 may be performed by the headset 125 before/during a hearing test, in accordance with the follow operations.

In some cases, the headset 125 may determine the user has an asymmetrical hearing ability. For instance, the headset 125 may determine the asymmetrical hearing ability based on a first set of one or more user responses from hearing testing (e.g., unoccluded data), and determine a second series of stimuli for hearing or fit testing based on a level of differential hearing. In some cases, the second series of stimuli for fit testing may include stimuli for a tested ear and masking stimuli for a non-testing ear.

In some cases, the headset 125 is configured to automatically change between air-conduction testing and bone-conduction testing based on user responses. Air- and bone-conduction threshold are used in the diagnosis of hearing loss. In some cases, the headset 125 may determine, based on user responses during hearing testing or fit resting, that masking is required to continue testing.

In operation 902, the headset 125 may determine a type of test. For instance, the headset 125 may retrieve an instruction message from the administrator device 105 (via the interface device 120) and extract a test ID or test indicator.

In operation O904, in response to determining the type of test is an air conduction test (O902: AC), the headset 125 may determine whether the headset 125 has BC thresholds (e.g., unmasked bone conduction thresholds for a pre-selected range of frequencies).

In operation O906, in response to determining the headset 125 has BC thresholds (O904: True), the headset 125 may determine whether TE threshold(s) (e.g., unmasked air conduction threshold for testing ear for a given frequencies) minus BC threshold(s) (for the same given frequencies) are greater than or equal to an interaural attenuation. Interaural attenuation (IA) may be a sound level at which playing sound to a test ear is so high that the non-test ear ear starts to hear the sound. Assuming you have a sensitive enough non-testing ear, the system may output a stimuli to the non-testing ear to mask the stimulus being output to the test ear.

In operation O908, in response to determining the headset 125 does not have BC thresholds (O904: False), the headset 125 may determine whether the TE threshold(s) are greater than or equal to IA.

In operation O910, in response to determining the type of test is a bone conduction test (O902: BC), the headset 125 may determine whether the TE threshold(s) minus BC threshold(s) are greater than 10 dB.

In operation O912, in response to operations O906, O908, or O910 returning True, the headset 125 may determine to apply a masking during the test. In operation O914, in response to operations O906, O908, or O910 returning False, the headset 125 may determine to not apply a masking during the test.

In operation O916, in response to determining to apply the masking during the test, the headset 125 may apply the masking during the test. To apply the masking during the test, the headset 125 may perform the following process (in pseudo-code):
(A) Set an initial masking level and an initial stimulus level:
   (1) Set initial masking level=NTE threshold (unmasked AC threshold, non-testing ear)+10 dB (or different offset value, such as 5 dB, 15 dB, and the like). If bone conducting testing, also add an occlusion effect factor, if necessary.
   (2) Set initial stimulus level=TE threshold.
   (3) Before starting the test, check if the (initial stimulus level−initial masking level)>IA. If so, set the initial masking level equal to the initial stimulus level−40 dB (or different offset value, such as 20 dB, 30 dB, and like).
(B) When subject does not respond:
   (1) If it is the first presentation of the test OR the user responded to the previous presentation, then the user gets a “second chance.” Keep the stimulus level and the masking level the same.
   (2) If the B1 does not apply, then:
      (i) Increase stimulus level by 5 dB (or different increase value, such as 2 dB, 7 dB, 10 dB, and the like).
      (ii) Increase masking level by 5 dB (or different increase value, such as 2 dB, 7 dB, 10 dB, and the like), if (masking level)>or=(stimulus level−IA+BC threshold).
(C) When the subject does respond:
   (1) Increase masking level by 5 dB (or different increase masking value, such as 2 dB, 7 dB, 10 dB, and the like). The stimulus level does not increase.
(D) The test ends when one of the following happens:
   (1) User responds three times in a row (including “second chance” responses). Result: masked threshold=stimulus level of last presentation.
   (2) The masking level exceeds 85 dB (or other max value, such as 80 dB, 90 dB, and the like). Result: Cannot determine masked threshold (system limitations). Flag for administrator.
   (3) The (masking level−IA)>BC threshold. Result: Cannot determine masked threshold (masking dilemma). Flag for administrator.

Thus, in some cases, masking may be automatically applied during air conduction or bone conduction testing, without intervention of the administrator.

### Accelerated Threshold Determination

FIG. 10 depicts a diagram for accelerated threshold determination. The features of the diagram for accelerated threshold determination of FIG. 10 may apply to FIGS. 1, 2A-2B, 3A-3E, 4A-4B, 5A-5C, 6A-6B, 7A-7B, 8A-8C, 9, 11A-11E, 12, and 13. The diagram depicts different series of presentations for a series of stimuli in accordance with different hearing testing processes. In particular, the diagram depicts a first series of stimuli 1002 presented in accordance with Hughson-Westlake procedures and a second series of stimuli 1004 presented in accordance with an accelerated threshold determination procedure.

Generally, Hughson-Westlake procedures may adjust stimulus levels (dB, for a given frequency) in a linear trend at a fixed interval (e.g. 15 dB, 10dB, or 5 dB) until a change in user response occurs, then the linear trend is reversed (and the fixed interval may be adjusted until the linear trend is reversed again). For instance, the first series of stimuli 1002 may depict: a first linear trend of presentations 1002A (response), 1002B (response), and 1000C (no response); then a trend reversal and a second linear trend of presentations 1002D (no response), 1002E (no response), and 1002F (response); then a trend reversal and a third linear trend of presentations 1002G (no response); and then a trend reversal and a fourth trend of presentations 1002H (no response), and 1002I (response). The first linear trend may have a fixed interval of 15 dB, the second linear trend may have a fixed interval of 5 dB, the third linear trend may have a fixed interval of 10 dB, and the fourth linear trend may have a fixed interval of 5 dB. In this manner, Hughson-Westlake procedures may discover hearing ability (e.g., threshold is 15 dB) of the user by linear trends of fixed intervals.

In contrast, the accelerated threshold determination procedure may adjust stimulus levels (dB, for a given frequency) in a variable trend at a variable interval until a change in user response occurs, thereby discovering the hearing ability of the user in a lower number of presentations. In some cases, the accelerated threshold determination procedure may determine a portion of the series of stimuli to be variably spaced across levels (e.g., an amplitude domain) for a given frequency.

For instance, the accelerated threshold determination procedure may make use of digital electronics to select variable levels (as opposed to a hardware electronics with a fixed minimum interval). For instance, the accelerated threshold determination procedure may start at presentation 1004A (response) in a high confidence level (e.g., 30 dB) for a frequency, then subsequently adjust the level based on response (or not) and variable intervals, such as presentation 1004B (10 dB, no response, interval −20 dB from previous presentation), presentation 1004C (20 dB, response, interval +10 dB from previous presentation), presentation 1004D (12 dB, no response, interval −8 dB from previous presentation), presentation 1004E (17 dB, response, interval +5 dB from previous presentation), presentation 1004F (13 dB, no response, interval −4 dB from previous presentation), and presentation 1004G (14 dB, response, interval +1 dB from previous presentation). In some cases, the variable interval may become smaller as user responses change, thus discovering the boundary of hearing ability faster as the procedure records different user responses. In the diagram, the accelerated threshold determination procedure may determine the hearing ability (e.g., threshold of hearing) is 13.5 dB, as the variable interval reduced in size from 20 dB down (from presentation 1004A to 1004B) to 1 dB (from presentation 1004F to 1004G). In some cases, the variable interval may present stimuli further away from the expected threshold to confirm that test taker responds as expected. For instance, the procedure may present stimuli further away from the expected threshold to engage the user and keep the user on task. In some cases, the procedure may numerically drop the results that are closest to the threshold to avoid a case where those responses may have been accidental (i.e., the user responded when they shouldn't have or failed to respond when they should have). As users may make mistakes, the procedure is designed to be robust to user error or mistake.

In this manner, each hearing test may reduce test time by avoiding additional presentations to discover the hearing threshold of the user. For instance, in the diagram, the accelerated threshold determination may save two presentations (˜5 to 10 seconds of test time) for a given frequency. While not a large time savings for a single test or user, over thousands or millions of tests the aggregate time saved may be substantial. The aggregate saved time may be especially useful for large organizations, such as manufacturing, military, or other high-noise risk occupations.

### Graphical User Interfaces for Testing

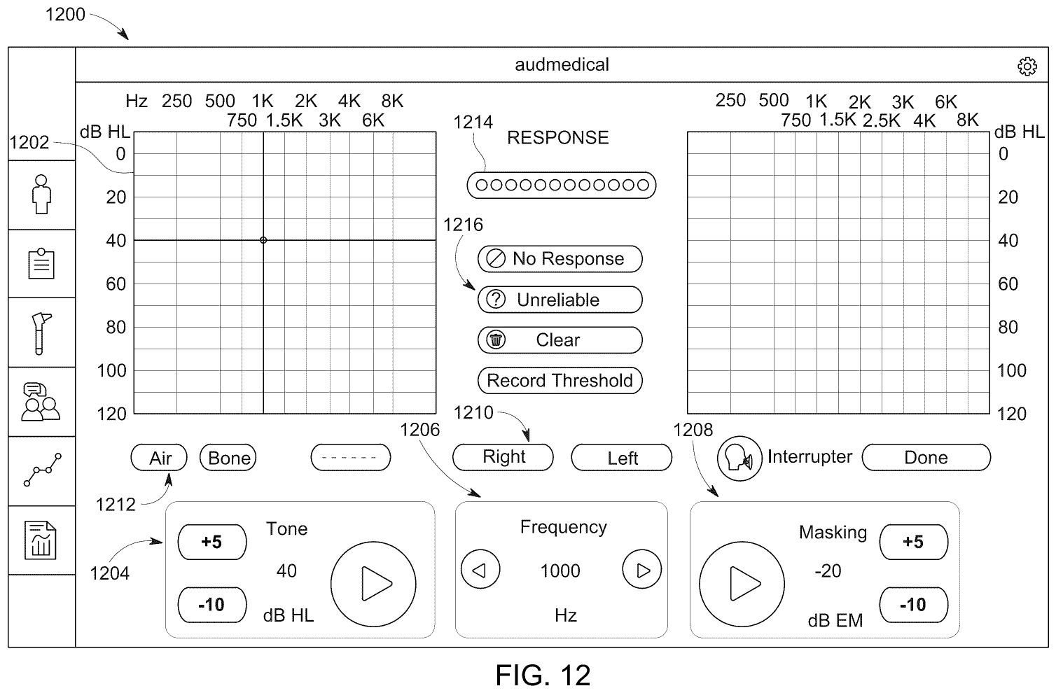

FIGS. 11A-11E and 12 depict graphic user interfaces on an administrator device 105 and an interface device 120. The graphic user interfaces on the administrator 105 device and the interface device 120 of FIGS. 11A-11E and 12 may apply to FIGS. 1, 2A-2B, 3A-3E, 4A-4B, 5A-5C, 6A-6B, 7A-7B, 8A-8C, 9, 10, and 13.

FIGS. 11A-11E depict diagrams 1100A, 1100B, 1100C, 1100D, and 1100E of graphical user interfaces for automated testing.

In the diagram 1100A, the administrator device 105 may output a sequence of graphical user interfaces. The graphical user interfaces may include a set up interface 1100A-1, a hardware check interface 1100A-2, and a calibration check interface 1100A-3.

The set up interface 1100A-1 may display connected interface device(s) 120 and connected headset(s) 125, in system fields 1106 and 1108, respectively. Thus, the administrator device 105 may be configured to manage a plurality of hearing or fit tests for the plurality of users. In some cases, the administrator device 105 may be configured to manage the plurality of hearing or fit tests in an asynchronous manner. In some cases, the administrator device 105 may be configured to manage the plurality of hearing or fit tests in a synchronous manner.

The system fields 1106 and 1108 may depict information about connected devices (e.g., battery life, connection strength, and the like) and UI elements to start a hardware check and/or a calibration check for individual connected devices. The set up interface 1100A-1 may also include UI elements to start a hardware check 1102 and/or a calibration check 1104 for all connected devices. In some cases, the set up interface 1100A-1 may also display warnings (if applicable) regarding connected devices. The system fields 1106 and 1108 may also include a test taker UI element 1107. The test taker UI element 1107 may be selectable by a user/administrator to select or input a user assigned to take a test with a headset 125.

The hardware check interface 1100A-2 maybe a hardware check interface. The administrator device 105 (or the interface device 120) may display the hardware check interface 1100A-2 in response to a user input selecting a UI element to start a hardware check (e.g., the hardware check 1102), e.g., on the administrator device 105. The hardware check interface 1100A-2 may display graphics (e.g., diagrams) and/or instructions to guide a user/administrator for hardware checks. The hardware check interface 1100A-2 may also include UI elements 1110, 1112, and 1114. The UI elements 1110, 1112, and 1114 may be selectable by a user/administrator to indicate a corresponding hardware component has been checked (e.g., UI element 1110 for cables, UI element 1112 for headband tension, and UI element 1114 for cushions). The administrator device 105 may log the hardware data in association with a test of a user and/or previous hardware data for the headset 125. In the case the hardware check interface 1100A-2 is displayed by the interface device 120, the user inputs to the UI elements 1110, 1112, and 1114 maybe transmitted to the administrator device 105 or stored locally (until being relayed later to the administrator device 105).

The calibration check interface 1100A-3 maybe a calibration check interface. The administrator device 105 (or the interface device 120) may display the calibration check interface 1100A-3 in response to a user input selecting a UI element to start a calibration check (e.g., the calibration check 1104), e.g., on the administrator device 105. The calibration check interface 1100A-3 may display graphics (e.g., data) and/or instructions to guide a user/administrator for calibration. The calibration check interface 1100A-3 may also include an UI element 1116. The UI element 1116 may be selectable by a user/administrator to initiate a calibration process. The administrator device 105 may log the current calibration data in association with a test of a user and/or previous calibration data for the headset 125. In the case the calibration check interface 1100A-3 is displayed by the administrator device 105, the user input to the UI element 1116 may cause the administrator device 105 to transmit an instruction message to the interface device 120, to thereby cause the interface device 120 to transmit an instruction message to the headset 125 to perform a calibration. In the case the calibration check interface 1100A-3 is displayed by the interface device 120, the user input to the UI element 1116 may cause the interface device 120 to transmit an instruction message to the headset 125 to perform a calibration. In response to receiving the instruction message to perform the calibration, the headset 125 may perform a defined sequence of actions to calibrate the headset 125, determine calibration data, and transmit the calibration data to the interface device 120. The interface device 120 may receive calibration data from the headset 125. The interface device 120 may then transmit calibration data to the administrator device 105 or store the calibration data locally (until being relayed later to the administrator device 105).

In the diagram 1100B, the administrator device 105 may display a test taker assignment interface 1100B-1 in response to a user input selecting the test taker UI element 1107. The test taker assignment interface 1100B-1 may display a list of users 1118 for testing. The list of users 1118 maybe selectable by a user input to indicate a user to test on a corresponding headset 125. The administrator device 105 may obtain the list of users based on user inputs or data from the server 115, and the like. In some cases, the administrator device 105 may input user data to assign the user to the headset 125 for a test. In response to a user being selected/inputted 1120 for a headset 125, the administrator device 105 may transmit an instruction message to the interface device 120 associated (e.g., connected to) the headset 125 for the test. The instruction message may include data of the user. The interface device 120 may then display a welcome interface 1100B-2.

The welcome interface 1100B-2 may display data (e.g., of the user) and/or instructions. The welcome interface 1100B-2 may also display a first confirmation UI element 1122 and a second confirmation UI element 1124. The first confirmation UI element 1122 maybe user selectable to indicate the user corresponds to the data displayed on the welcome interface 1100B-2. The second confirmation UI element 1124 maybe user selectable to indicate the user does not correspond to the data displayed on the welcome interface 1100B-2. The administrator device 105 may log the confirmation (or not) in association with a test of a user. For instance, the interface device 120 may transmit the confirmation data to the administrator device 105 or store the confirmation data locally (until being relayed later to the administrator device 105).

In the diagram 1100C, the administrator device 105 may display a first evaluation interface 1100C-1 and then a second evaluation interface 1100C-2. The first evaluation interface 1100C-1 may display a list of tests 1126 that the headset 125 may be configured to perform for the user. The list of tests 1126 maybe user selectable to select a test to perform 1128. In response to a user/administrator input selecting the test to perform 1128, the administrator device 105 may proceed to display the test the second evaluation interface 1100C-2 and transmit an instruction message to the interface device 120. The instruction message may indicate the selected test to perform 1128 and any relevant data (e.g., previous tests, user data, and the like). The interface device 120 may receive, process, and relay the instruction message to the headset 125. For instance, the interface device 120 may display the test interface 604 (with the start/pause UI element 604A and the response UI element 604B). In some cases, the interface device 120 may display the test interface 604 after a user input indicated the user is ready or after the headset 125 transmitted an acknowledgement that the headset 125 is ready. The interface device 120 may in response to a user input selecting the start/pause UI element 604A (e.g., to start the test), transmit an instruction to the headset 125 to start the test. The interface device 120 may in response to a user input on either the start/pause UI element 604A or the response UI element 604B, transmit a message to the headset 125. The headset 125 may start/pause the test in response to the message indicating a start/pause. The headset 125 may record a response in response to the message indicating a response to the stimulus. The headset 125 and the interface device 120 may cooperate to complete the selected test (e.g., fit or hearing test) by the headset 125 outputting stimuli (e.g., sound outputs for air conduction testing or vibration outputs for bone conduction testing) and the interface device 120 transmitting responses based on user interaction with the response UI element 604B to the headset 125. After the selected test is completed, the headset 125 may transmit a final result message to the interface device 120. The final result message may include data and results (“test dataset”) of the selected test, such as an indication that a fit test was passed (e.g., sufficient attenuation) or failed (e.g., insufficient attenuation).

The second evaluation interface 1100C-2 may display connected interface device(s) 120 and connected headset(s) 125 that are performing tests with users, in system fields 1130 and 1132, respectively. The system fields 1130 and 1132 may depict information about connected devices (e.g., battery life, connection strength, and the like) and UI elements. The system fields 1130 and 1132 may also include a status indicator 1134 and communication UI element 1136.

The status indicator 1134 may indicate a progress of a test for a particular user/headset 125. The administrator device 105 may update the status indicator 1134 based on intermediate results transmitted from the headset 125, via the interface device 120. In some cases, the status indicator 1134 may indicate milestones of the test (e.g., training, in test, test completed). In some cases, the status indicator 1134 (or the system fields 1130 and 1132) may indicate particular data regarding aspects of the test the user is currently performing (e.g., a series of stimuli, responses, response times, and the like).

The communication UI element 1136 maybe user/administrator selectable to launch a communication application. The communication application may be a text and/or voice communication interface (e.g., one or the other, or the administrator can toggle) between the administrator device 105, the interface device 120, and the headset 125. In the case the communication application is configured for text communication, the administrator device 105 may transmit/receive/display text messages to/from the interface device 120, and the interface device 120 may transmit/receive/display text messages to/from the administrator device 105. In the case the communication application is configured for voice communication, the administrator device 105 may transmit voice data to the headset 125, via the interface device 120, to instruct, inform, or alert the user. In some cases, the headset 125 may automatically listen (e.g., in response to receiving voice data from the interface device, via a microphone) for user voice responses, and transmit voice data to the administrator device 105, via the interface device 120.

In some cases, the interface device 120, the headset 125, and the administrator device 105 form wireless connections using Bluetooth or WIFI communications. In some cases, the administrator device 105 may be configured to remotely connected to the interface device 120 via a secure server connection (via server 115). In some cases, the administrator device 105 may be configured to connected to the interface device 120 via a direct WIFI connection.

In some cases, the wireless connections between the interface device 120, the headset 125, and the administrator device 105 are Bluetooth low energy connections. In the case the communication application is configured for voice communication, the administrator device 105 may be configured to, in response to receiving an administrator user input on the administrator device 105 for voice transmission to the headset 125, change the wireless connections to standard Bluetooth communications (from Bluetooth low energy). The administrator device 105 may proceed to transmit voice data over the standard Bluetooth communications to the headset 125, via the interface device 120.

In the diagram 1100D, the interface device 120 may display a first result interface 1100D-1 or a second result interface 1100D-2. The interface device 120 may display the first result interface 1100D-1 or the second result interface 1100D-2 based on the final result message received from the headset 125. The first result interface 1100D-1 may correspond to a hearing test and display data and/or graphics regarding the hearing test (e.g. stimuli presented, user responses, thresholds, and the like). The second result interface 1100D-2 may correspond to a fit test and display data and/or graphics regarding the fit test (e.g. stimuli presented, user responses, thresholds, frequency attenuation, and the like) and an indicator of sufficient (or not) attenuation of a hearing protector. Both of the first result interface 1100D-1 and the second result interface 1100D-2 may include a signature UI element 1138. The signature UI element 1138 may be user selectable to input a mark (e.g., stylus or text input) to confirm the user was the user for this test. In response to a user input on the signature UI element 1138, the interface device 120 may transmit signature data to the administrator device 105. The administrator device 105 may log the signature data and the test dataset (reported with or separately from the signature data) in association with the test of the user. For instance, the interface device 120 may transmit the signature data and the test dataset to the administrator device 105 or store the signature data and the test dataset locally (until being relayed later to the administrator device 105).

In the diagram 1100E, the administrator device 105 may display a report interface 1100E-1 and the interface device 120 may display a finish test interface 1100E-2. For instance, the interface device 120 may display the finish test interface 1100E-2 in response to the user input on the signature UI element 1138. The administrator device 105 may display the report interface 1100E-1 in response to receiving the signature data and/or the test dataset. The report interface 1100E-1 may include a list of report types 1140. Each report type of the list of report types 1140 may display data (e.g., status of report, number of reports, and like) and options for managing the report, the test dataset, and any associated log data (collectively, “session data”). For instance, the administrator device 105 may erase the session data from the administrator device 105/interface device 120 after the session data is sent to the server 115 for record keeping/auditing purposes. The report interface 1100E-1 may also include an export UI element 1142. The export UI element 1142 may be user selectable to cause the administrator device 105 to transmit the session data (when ready) to the server 115 or a different endpoint (e.g., organization, patient/user, cloud provider, and the like).

FIG. 12 depicts a manual test interface 1200 for administrator testing. The administrator device 105 may display the manual test interface 1200 in response to selecting a manual administrator test. For instance, the user/administrator may select a manual test in a same location as a user (e.g., in a same room/geographic location) or remotely from the user, and manually make frequency/level selections for stimuli, masking, testing ear, non-testing ear, and the like.

The manual test interface 1200 may include a graphic element 1202, stimulus level element 1204, a frequency element 1206, a masking element 1208, a testing ear element 1210, a test type element 1212, a response time indicator element 1214, and a response type indicator element 1216.

The stimulus level element 1204 may be user selectable to adjust a level (in dB) of a stimulus. The frequency element 1206 may be user selectable to adjust a frequency (in Hz) of a stimulus. The graphic element 1202 may display a currently selected level and frequency on a chart. In some cases, the graphic element 1202 may display previous presentations of stimulus (and response or not). In some cases, the graphic element 1202 may be user selectable to adjust a level and frequency by dragging or touching (or clicking) on a region of the chart. The masking element 1208 may be user selectable to adjust a level (in dB) of a masking stimuli of a non-testing ear (if toggled on). The testing ear element 1210 may be user selectable to toggle which ear is the testing ear and which ear is the non-testing ear. The test type element 1212 may be user selectable to toggle between air conduction testing and bone conducting testing (if the bone conduction headset is used). Based on a user input (or, e.g., a time elapse) the administrator device 105 may transmit an instruction to the headset 125, via the interface device 120, to output a stimulus in accordance with the selections input by the administrator. The headset 125 may receive the instruction and output a stimulus (and any masking) in accordance with the instruction. The interface device 120 may receive a user response (if the user selects the response UI element 604B) and transmit a response message to the headset 125. The headset 125 may determine whether the user responded to the stimulus (e.g., if it receives a response message indicating the user responded within a response window), and transmit a result message to the administrator device 105, via the interface device 120. The administrator device 105 may receive the result message.

The administrator device 105 may determine a response time and response type based on the result message. The response time indicator element 1214 may display the response time and the response type indicator element 1216 may display the response type (response, no response, unreliable, etc.). The response type indicator element 1216 may also include an UI element selectable by the administrator to record a threshold based on the response. In this manner, the administrator using the administrator device 105 may observe a response time/response type even if a network lag/connection degrades a video or teleconference connection. Once the manual test is complete, the administrator device 105 may transmit a test complete instruction to the headset 125 and the interface device 120, so the user/headset 125/interface device 120 is aware the test is complete. Similar graphic user interfaces as discussed above with respect to FIGS. 11A-11E may be used before, during, and after the manual test.

### Computer System

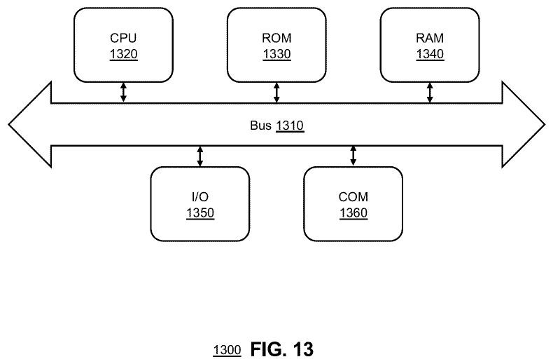

FIG. 13 depicts an example system that may execute techniques presented herein. FIG. 13 is a simplified functional block diagram of a computer that may be configured to execute techniques described herein, according to exemplary cases of the present disclosure. Specifically, the computer (or “platform” as it may not be a single physical computer infrastructure) may include a data communication interface 1360 for packet data communication. The platform may also include a central processing unit (“CPU”) 1320, in the form of one or more processors, for executing program instructions. The platform may include an internal communication bus 1310, and the platform may also include a program storage and/or a data storage for various data files to be processed and/or communicated by the platform such as ROM 1330 and RAM 1340, although the system 1300 may receive programming and data via network communications. The system 1300 also may include input and output ports 1350 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In some cases, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EPSOM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

### Terminology

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

### EXAMPLES

Exemplary embodiments of the systems and methods disclosed herein are described in the numbered paragraphs below.
A1. A system for hearing testing and fit testing, the system comprising:
   a headset, wherein the headset includes:
      a support structure; and
      a pair of ear cups connected by the support structure, wherein each of the pair of ear cups include:
         a cushion configured to seal an interior of the ear cup from an exterior of the ear cup when worn by a user;
         an audiometer stack disposed in the interior of the ear cup; and
         an ear cup attenuating structure surrounding the audiometer stack and being connected to the cushion, wherein the ear cup attenuating structure is configured to provide attenuation comparable to that of a sound booth; and
   an interface device configured to wirelessly connect to at least a first audiometer stack of the pair of ear cups;
   wherein the system is configured to:
      output a first interface, the first interface including a first interface element configured to be selectable by a first input to start a hearing test;
      determine that a first input has selected the first interface element;
      in response to determining the first input has selected the first interface element, instruct at least the first audiometer stack to perform a first series of stimuli;
      receive a first set of one or more user responses to at least a first subset of the first series of stimuli;
      based on the first set of one or more user responses, determine a hearing test result of the hearing test, the hearing test result including at least a measure of a hearing ability of the user;
      output a second interface configured to indicate a transition to a hearing protector fit test, the second interface including at least a second interface element configured to be selectable by a second input to start the hearing protector fit test;
      determine a second input has selected the second interface element;
   in response to determining the second input has selected the second interface element, instruct at least the first audiometer stack to perform a second series of stimuli;
      receive a second set of one or more user responses for at least a second subset of the second series of stimuli; and
      based on the second set of one or more user responses, determine a fit test result of the hearing protector fit test, the fit test result including an indication of a hearing attenuation achieved by a hearing protector as worn by the user during the hearing protector fit test.
A2. The system of A1, wherein the interface device is a patient device or an administrator device.
A3. The system of any of A1-A2, wherein the first interface is configured to instruct the user on how to use the system.
A4. The system of any of A1-A3, wherein each audiometer stack includes at least: a speaker and a mounting structure to mount the speaker within the ear cup.
A5. The system of A4, wherein the first audiometer stack is connected to a second audiometer stack of the pair of ear cups.
A6. The system of A5, wherein the first audiometer stack includes control electronics and a wireless communication transceiver.
A7. The system of A6, wherein the control electronics are configured to receive, via the wireless communication transceiver, interface device instructions from the interface device and transmit analog signals to each speaker of the first and second audiometer stack.
A8. The system of A6, wherein the control electronics are configured to transmit, via the wireless communication transceiver, data to the user device regarding presented stimuli and any user responses.
A9. The system of A5, wherein the second audiometer stack includes a battery configured to power each of the first and second audiometer stack.
A10. The system of A4, wherein the mounting structure is positioned inside the interior of the ear cup so as to provide a clearance between (a) the speaker and mounting structure and (b) hearing protectors and/or user's ears when applied to the user.
A11. The system of any of A1-A10, wherein each ear cup attenuating structure includes insulation and a case.
A12. The system of A11, wherein the insulation is configured to attenuate high frequency noise from the exterior of the ear cup.
A13. The system of A11, wherein the case is configured to attenuate low frequency noise from the exterior of the ear cup.
A14. The system of A13, wherein the case includes a side wall extending away from the cushion and an end wall to enclose the side wall, the end wall being curved in at least one dimension to reduce resonance with the stimuli from a speaker of the audiometer stack.
A15. The system of any of A1-A14, wherein the first set of one or more user responses are based on inputs from the user on a patient device or inputs from an administrator on an administrator device.
A16. The system of any of A1-A15, wherein the system is further configured to: determine a portion of the first series of stimuli based on a portion of the first set of one or more user responses.
A17. The system of any of A1-A16, wherein the system is further configured to: determine the second series of stimuli based on the first set of one or more user responses, and determine the fit test result based on the first set of one or more user responses and the second set of one or more user responses.
A18. The system of any of A1-A17, wherein the system is further configured to determine the user has a greater than a threshold level of asymmetrical hearing ability based on the first set of one or more user responses, and determine the second series of stimuli based on a level of differential hearing.
A19. The system of any of A1-A18, wherein the system is further configured to, before determining the fit test result of the hearing protector fit test and while performing the second series of stimuli: determine, for a user response responsive to a specific frequency and amplitude, that the hearing protector is improperly applied to the user; and output an instruction to adjust the hearing protector before continuing the hearing protector fit test.
A20. A method for hearing testing and fit testing, the method comprising:
   output a first interface, the first interface including a first interface element configured to be selectable by a first input to start a hearing test;
   determine that a first input has selected the first interface element;
   in response to determining the first input has selected the first interface element, instruct at least a first audiometer stack of a pair audiometer stacks to perform a first series of stimuli;
   receive a first set of one or more user responses for at least a first subset of the first series of stimuli;
   based on the first set of one or more user responses, determine a hearing test result of the hearing test, the hearing test result including at least a measure of a hearing ability of the user;
   output a second interface configured to indicate a transition to a hearing protector fit test, the second interface including at least a second interface element configured to be selectable by a second input to start the hearing protector fit test;
   determine a second input has selected the second interface element;
   in response to determining the second input has selected the second interface element, instruct at least the first audiometer stack to perform a second series of stimuli;
   receive a second set of one or more user responses for at least a second subset of the second series of stimuli; and
   based on the second set of one or more user responses, determine a fit test result of the hearing protector fit test, the fit test result including an indication of a sound attenuation achieved by a hearing protector as worn by the user during the hearing protector fit test.
B1. A system for bone conduction hearing or fit testing, the system comprising:
   a headset, wherein the headset includes:
      a support structure;
      a pair of ear cups connected by the support structure, wherein each of the pair of ear cups include:
         a cushion configured to seal an interior of the ear cup from an exterior of the ear cup when worn by a user;
         an audiometer stack disposed in the interior of the ear cup; and
         an ear cup attenuating structure surrounding the audiometer stack and being connected to the cushion, wherein the ear cup attenuating structure is configured to provide attenuation comparable to that of a sound booth; and
      a bone conductor configured to be mounted to a head of the user and electronically connected to a first audiometer stack of the pair of ear cups, wherein a second audiometer stack of the pair of ear cups is electronically connected to the first audiometer stack; and
   an interface device configured to wirelessly connect to the first audiometer stack and transmit instructions to and receive data from the first audiometer stack;
   wherein the first audiometer stack includes a first analog output, a second analog output, and a third analog output,
   the first analog output drives a first speaker associated with the first audiometer stack,
   the second analog output drives a second speaker associated with the second audiometer stack,
   the third analog output drives the bone conductor, and
   the third analog output is different than the first analog output and the second analog output.
B2. The system of B1, wherein the first analog output, the second analog output, and the third analog output are each configured to output a respective dynamic range.
B3. The system of B2, wherein the first analog output, the second analog output, and the third analog output are configured to be driven separately.
B4. The system of B2, wherein the third analog output has a different dynamic range than the first analog output and the second analog output.
B5. The system of B2, wherein the dynamic range of the third analog output is smaller than the dynamic range of the first analog output and the second analog output.
B6. The system of B2, wherein the dynamic range of the third analog output has a lower upper range than the dynamic range of the first analog output and the second analog output.
B7. The system of any of B1-B6, wherein the support structure of the headset is a first support structure, and the bone conductor includes a second support structure.
B8. The system of B7, wherein the second support structure is configured to hold the bone conductor on the head of the user.
B9. The system of B7, wherein the second support structure is configured to be adjustable to apply a variable fit and/or an amount of contact pressure between the bone conductor and the head of the user.
B10. The system of B7, wherein the bone conductor includes a pressure monitor.
B11. The system of B10, wherein the pressure monitor is configured to indicate a threshold contact pressure for hearing/fit testing has been applied between the bone conductor and the head of the user.
B12. The system of any of B1-B11, wherein the system is configured to: automatically change between air-conduction testing and bone-conduction testing based on user responses.
B13. The system of B12, wherein the system is configured to: determine, based on user responses, masking is required to continue testing.
B14. A method for hearing testing and fit testing, the method comprising:
   wirelessly connect an interface device to a first audiometer stack of a headset, to thereby transmit instructions to and receive data from the first audiometer stack,
      wherein the first audiometer stack of the headset is associated with a first ear cup of a pair of ear cups of the headset,
      a second audiometer stack of the headset is associated with a second ear cup of the pair of ear cups of the headset,
      the second audiometer stack is electronically connected to the first audiometer stack, and
      each of the pair of ear cups include:
         a cushion configured to seal an interior of the ear cup from an exterior of the ear cup when worn by a user, and
         an ear cup attenuating structure surrounding a respective audiometer stack and being connected to the cushion, wherein the ear cup attenuating structure is configured to provide attenuation comparable to that of a sound booth;
   transmit an instruction message to the first audiometer stack of the headset, the instruction message indicating an instruction to start a hearing or fit test of a user, wherein the first audiometer stack includes a first analog output, a second analog output, and a third analog output;
   in accordance a firmware of the first audiometer stack, drive, via the first analog output, a first speaker associated with the first audiometer stack and the first ear cup;
   in accordance the firmware of the first audiometer stack, drive, via the second analog output, a second speaker associated with the second audiometer stack and the second ear cup; and
   in accordance the firmware of the first audiometer stack, drive, via the third analog output, a bone conductor of the headset, wherein the bone conductor is configured to be mounted to a head of the user and electronically connected to the first audiometer stack;
   wherein the third analog output is different than the first analog output and the second analog output.
B15. The method of B14, wherein the first analog output, the second analog output, and the third analog output are each configured to output a respective dynamic range.
B16. The method of B15, wherein the first analog output, the second analog output, and the third analog output are configured to be driven separately.
B17. The method of B15, wherein the third analog output has a different dynamic range than the first analog output and the second analog output.
B18. The method of B15, wherein the dynamic range of the third analog output is smaller than the dynamic range of the first analog output and the second analog output.
B19. The method of B15, wherein the dynamic range of the third analog output has a lower upper range than the dynamic range of the first analog output and the second analog output.
B20. The method of and of B14-B19, further comprising: automatically change between air-conduction testing and bone-conduction testing based on user feedback.
C1. A system for hearing or fit testing, the system comprising:
   at least one headset, wherein the least one headset includes a first headset, the first headset includes:
      a support structure; and
      a pair of ear cups connected by the support structure, wherein each of the pair of ear cups include:
         a cushion configured to seal an interior of the ear cup from an exterior of the ear cup when worn by a user;
         an audiometer stack disposed in the interior of the ear cup; and
         an ear cup attenuating structure surrounding the audiometer stack and being connected to the cushion, wherein the ear cup attenuating structure is configured to provide attenuation comparable to that of a sound booth;
   at least one interface device, wherein the least one interface device includes a first interface device configured to wirelessly connect to at least a first audiometer stack of the first headset; and
   an administrator device configured to wirelessly connect to each of the at least one interface device, to thereby transmit instructions to and receive data from each of the at least one headset via the at least one interface device,
   wherein the administrator device is configured to, in response to receiving an administrator user input on the administrator device, transmit an instruction message, via the first interface device, to the first headset to conduct a hearing or fit test, and
   the instruction message does not include data indicating frequency of stimuli, amplitude of stimuli, or timing of stimuli.
C2. The system of C1, wherein the administrator device is configured to receive, via the first interface device, data indicating status of the hearing or fit test.
C3. The system of C2, wherein the data indicating status of the hearing or fit test is generated and transmitted from the first headset.
C4. The system of C1, wherein the administrator device is configured to receive, via the first interface device, data indicating response time(s) of a user of the first headset during the hearing or fit test.
C5. The system of C4, wherein the data indicating response time(s) of a user of the first headset during the hearing or fit test is generated and transmitted from the first headset.
C6. The system of C4, wherein the first headset determines the response time(s) by a clock determining a difference from when a first stimulus is output by first or second speaker of the first headset and when the user interacts with the first interface device.
C7. The system of any of C1-C6, wherein the at least one headset comprises a plurality of headsets for use by a plurality of users, and the administrator device is configured to manage a plurality of hearing or fit tests for the plurality of users.
C8. The system of C7, wherein the administrator device is configured to manage the plurality of hearing or fit tests in an asynchronous or synchronous manner.
C9. The system of any of C1-C8, wherein the first interface device, the first headset, and the administrator device form wireless connections using Bluetooth or WIFI communications.
C10. The system of C9, wherein the administrator device is configured to remotely connected to the first interface device via a secure server connection.
C11. The system of C9, wherein the administrator device is configured to connected to the first interface device via a direct WIFI connection.
C12. The system of C9, wherein the wireless connections between the first interface device, the first headset, and the administrator device are Bluetooth low energy connections.
C13. The system of C12, wherein the administrator device is configured to, in response to receiving an administrator user input on the administrator device for voice transmission to the first headset, change the wireless connections to standard Bluetooth communications, and transmit voice data over the standard Bluetooth communications to the first headset.
C14. The system of any of C1-C13, wherein the first headset includes firmware, a voltage module, and at least two analog outputs.
C15. The system of C14, wherein the firmware is configured to determine the frequency of stimuli, the amplitude of stimuli, and the timing of stimuli (collectively, test parameters) based on a type of hearing or fit test being performed.
C16. The system of C14, wherein the firmware adjusts the test parameters based on user response and/or response time.
C17. The system of C14, wherein the voltage module is configured to output a dynamic range for each of the at least two analog outputs.
C18. The system of C17, wherein an output of the dynamic range is configured to be a pure tone or range of frequencies.
C19. The system of C18, wherein the pure tone is a narrow bandwidth range of frequency.
C20. A method for hearing or fit testing, the method comprising:
wirelessly connect at least one interface device to at least one headset; wherein the least one interface device includes a first interface device configured to wirelessly connect to at least a first audiometer stack of a first headset;
wirelessly connect an administrator device to each of the at least one interface device, to thereby transmit instructions to and receive data from each of the at least one headset via the at least one interface device; and
in response to receiving an administrator user input on the administrator device, transmit an instruction message, via the first interface device, to the first headset to conduct a hearing or fit test,
wherein the instruction message does not include data indicating frequency of stimuli, amplitude of stimuli, or timing of stimuli.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for bone conduction hearing or fit testing, the system comprising:
a headset, wherein the headset includes:
a support structure;
a pair of ear cups connected by the support structure, wherein each of the pair of ear cups include:
a cushion configured to seal an interior of the ear cup from an exterior of the ear cup when worn by a user;
an audiometer stack disposed in the interior of the ear cup; and
an ear cup attenuating structure surrounding the audiometer stack and being connected to the cushion, wherein the ear cup attenuating structure is configured to provide attenuation comparable to that of a sound booth; and
a bone conductor configured to be mounted to a head of the user and electronically connected to a first audiometer stack of the pair of ear cups, wherein a second audiometer stack of the pair of ear cups is electronically connected to the first audiometer stack; and
an interface device configured to wirelessly connect to the first audiometer stack and transmit instructions to and receive data from the first audiometer stack;
wherein the first audiometer stack includes a first analog output, a second analog output, and a third analog output,
the first analog output drives a first speaker associated with the first audiometer stack,
the second analog output drives a second speaker associated with the second audiometer stack,
the third analog output drives the bone conductor, and
the third analog output is different than the first analog output and the second analog output.

2. The system of claim 1, wherein the first analog output, the second analog output, and the third analog output are each configured to output a respective dynamic range.

3. The system of claim 2, wherein the first analog output, the second analog output, and the third analog output are configured to be driven separately.

4. The system of claim 2, wherein the third analog output has a different dynamic range than the first analog output and the second analog output.

5. The system of claim 2, wherein the dynamic range of the third analog output is smaller than the dynamic range of the first analog output and the second analog output.

6. The system of claim 2, wherein the dynamic range of the third analog output has a lower upper range than the dynamic range of the first analog output and the second analog output.

7. The system of claim 1, wherein the support structure of the headset is a first support structure, and the bone conductor includes a second support structure.

8. The system of claim 7, wherein the second support structure is configured to hold the bone conductor on the head of the user.

9. The system of claim 7, wherein the second support structure is configured to be adjustable to apply a variable fit and/or an amount of contact pressure between the bone conductor and the head of the user.

10. The system of claim 7, wherein the bone conductor includes a pressure monitor.

11. The system of claim 10, wherein the pressure monitor is configured to indicate a threshold contact pressure for hearing/fit testing has been applied between the bone conductor and the head of the user.

12. The system of claim 1, wherein the system is configured to: automatically change between air-conduction testing and bone-conduction testing based on user responses.

13. The system of claim 12, wherein the system is configured to: determine, based on user responses, masking is required to continue testing.

14. A method for bone conduction hearing or fit testing, the method comprising:
wirelessly connect an interface device to a first audiometer stack of a headset, to thereby transmit instructions to and receive data from the first audiometer stack,
wherein the first audiometer stack of the headset is associated with a first ear cup of a pair of ear cups of the headset,
a second audiometer stack of the headset is associated with a second ear cup of the pair of ear cups of the headset,
the second audiometer stack is electronically connected to the first audiometer stack, and
each of the pair of ear cups include:
a cushion configured to seal an interior of the ear cup from an exterior of the ear cup when worn by a user, and
an ear cup attenuating structure surrounding a respective audiometer stack and being connected to the cushion, wherein the ear cup attenuating structure is configured to provide attenuation comparable to that of a sound booth;
transmit an instruction message to the first audiometer stack of the headset, the instruction message indicating an instruction to start a hearing or fit test of a user, wherein the first audiometer stack includes a first analog output, a second analog output, and a third analog output;
in accordance a firmware of the first audiometer stack, drive, via the first analog output, a first speaker associated with the first audiometer stack and the first ear cup;
in accordance the firmware of the first audiometer stack, drive, via the second analog output, a second speaker associated with the second audiometer stack and the second ear cup; and
in accordance the firmware of the first audiometer stack, drive, via the third analog output, a bone conductor of the headset, wherein the bone conductor is configured to be mounted to a head of the user and electronically connected to the first audiometer stack;
wherein the third analog output is different than the first analog output and the second analog output.

15. The method of claim 14, wherein the first analog output, the second analog output, and the third analog output are each configured to output a respective dynamic range.

16. The method of claim 15, wherein the first analog output, the second analog output, and the third analog output are configured to be driven separately.

17. The method of claim 15, wherein the third analog output has a different dynamic range than the first analog output and the second analog output.

18. The method of claim 15, wherein the dynamic range of the third analog output is smaller than the dynamic range of the first analog output and the second analog output.

19. The method of claim 15, wherein the dynamic range of the third analog output has a lower upper range than the dynamic range of the first analog output and the second analog output.

20. The method of claim 14, further comprising: automatically change between air-conduction testing and bone-conduction testing based on user feedback.
